(19) United States
(12) Patent Application Publication
(10) Pub. No.: US 2021/0407077 A1
(43) Pub. Date: Dec. 30, 2021

(54) INFORMATION PROCESSING DEVICE AND MODEL GENERATION METHOD

(71) Applicant: HOYA CORPORATION, Tokyo (JP)

(72) Inventor: Takao MAKINO, Tokyo (JP)

(73) Assignee: HOYA CORPORATION, Tokyo (JP)

(21) Appl. No.: 17/252,942

(22) PCT Filed: Nov. 13, 2019
(86) PCT No.: PCT/JP2019/044578

§ 371 Date: Dec. 16, 2020

Related U.S. Application Data
(60) Provisional application No. 62/775,197, filed on Dec. 4, 2018.

(30) Foreign Application Priority Data
May 29, 2019 (JP) 2019-100647
May 29, 2019 (JP) 2019-100648
May 29, 2019 (JP) 2019-100649

(51) Int. Cl. G06T 7/00; A61B 1/04; G16H 50/20

(52) U.S. Cl. CPC G06T 7/0012; A61B 1/04; G16H 50/20; G06T 2207/30028; G06T 2207/20081; G06T 2207/20084; G06T 2207/10068

(57) ABSTRACT

To provide an information processing device and the like for presenting a determination reason together with a determination result regarding a disease. The information processing device includes: an image acquisition unit that acquires an endoscope image; a first acquisition unit that inputs the endoscope image acquired by the image acquisition unit to a first model that outputs diagnosis criteria prediction regarding diagnostic criteria of disease when the endoscope image is input, and acquires the output diagnosis criteria prediction; and an output unit that outputs the diagnosis criteria prediction acquired by the first acquisition unit in association with the diagnosis prediction regarding a state of the disease acquired based on the endoscope image.

### TECHNICAL FIELD

The present invention relates to an information processing device and a model generation method.

### BACKGROUND ART

An image processing device that performs texture analysis such as an endoscope image and classifies an image according to pathological diagnosis has been proposed. By using such a diagnosis support technique, even a doctor who does not have highly specialized knowledge and experience can perform a diagnosis promptly.

### CITATION LIST

### Patent Literature

Patent Literature 1: JP 2017-70609 A

### SUMMARY OF INVENTION

### Technical Problem

However, the classification by the image processing device of Patent Literature 1 is a black box for the user. Therefore, the user may not always understand and be convinced of the reason for the output classification.

For example, in ulcerative colitis (UC), it is known that determinations by specialists who look at the same endoscope image may be different. In the case of such a disease, there is a possibility that a doctor who is a user cannot understand the determination result by the diagnosis support technique.

In one aspect, an object of the present invention is to provide an information processing device or the like that presents a determination reason as well as a determination result regarding disease.

### Solution to Problem

An information processing device includes: an image acquisition unit that acquires an endoscope image; a first acquisition unit that inputs an endoscope image acquired by the image acquisition unit to a first model for outputting a diagnosis criteria prediction regarding a diagnosis criteria of a disease upon input of the endoscope image and that acquires the output diagnosis criteria prediction; and an output unit that outputs the diagnosis criteria prediction acquired by the first acquisition unit in association with diagnosis prediction regarding a state of the disease acquired on the basis of the endoscope image.

### Advantageous Effects of Invention

It is possible to provide the information processing device or the like that presents the region that contributes to the determination together with the determination result related to the diagnosis of the disease.

### BRIEF DESCRIPTION OF DRAWINGS

FIG. 1 is an explanatory diagram for explaining an outline of a diagnostic support system.

FIG. 2 is an explanatory diagram for explaining a configuration of the diagnostic support system.

FIG. 3 is an explanatory diagram for explaining a configuration of a first score learning model.

FIG. 4 is an explanatory diagram for explaining a configuration of a second model.

FIG. 5 is a time chart for schematically explaining an operation of a diagnostic support system.

FIG. 6 is a flowchart for explaining a process flow of a program.

FIG. 7 is an explanatory diagram for explaining an outline of a diagnostic support system according to a first modification.

FIG. 8 is an explanatory diagram for explaining a screen display according to a second modification.

FIG. 9 is an explanatory diagram for explaining a screen display according to a third modification.

FIG. 10 is a time chart for schematically explaining an operation of a fourth modification.

FIG. 11 is an explanatory diagram for explaining an outline of a process of generating a model.

FIG. 12 is an explanatory diagram for explaining a configuration of a model generation system.

FIG. 13 is an explanatory diagram for explaining a record layout of a training data DB.

FIG. 14 is an explanatory diagram for explaining a training data input screen.

FIG. 15 is an explanatory diagram for explaining the training data input screen.

FIG. 16 is a flowchart for explaining a process flow of a program that generates a learning model.

FIG. 17 is a flowchart for explaining a process flow of a program that updates a learning model.

FIG. 18 is a flowchart illustrating a process flow of a program that collects the training data.

FIG. 19 is an explanatory diagram for explaining an outline of a diagnostic support system according to a third embodiment.

FIG. 20 is an explanatory diagram for explaining a feature quantity acquired from a second model.

FIG. 21 is an explanatory diagram for explaining a conversion between a feature quantity and a score.

FIG. 22 is an explanatory diagram for explaining a record layout of a feature quantity DB.

FIG. 23 is a flowchart for explaining a process flow of a program that creates a converter.

FIG. 24 is a flowchart for explaining a process flow of a program during endoscope inspection according to a third embodiment.

FIG. 25 is an explanatory diagram for explaining an outline of a diagnostic support system according to a fourth embodiment.

FIG. 26 is a flowchart for explaining a conversion between an endoscope image and a score according to the fourth embodiment.

FIG. 27 is a flowchart for explaining a process flow of a program that creates the converter according to the fourth embodiment.

FIG. 28 is a flowchart for explaining a process flow of a program during the endoscope inspection according to the fourth embodiment.

FIG. 29 is an explanatory diagram for explaining an outline of a diagnostic support system according to a fifth embodiment.

FIG. 30 is an explanatory diagram for explaining a configuration of a first score learning model according to a sixth embodiment.

FIG. 31 is an explanatory diagram for explaining a screen display according to the sixth embodiment.

FIG. 32 is an explanatory diagram for explaining a screen display according to a seventh embodiment.

FIG. 33 is an explanatory diagram for explaining an outline of a diagnostic support system according to an eighth embodiment.

FIG. 34 is an explanatory diagram for explaining an outline of a diagnostic support system according to a ninth embodiment.

FIG. 35 is an explanatory diagram for explaining a configuration of the first model.

FIG. 36 is an explanatory diagram for explaining a configuration of an extraction unit.

FIG. 37 is a flowchart for explaining a process flow of a program according to a ninth embodiment.

FIG. 38 is a flowchart for explaining a process flow of a subroutine of an area of interest extraction.

FIG. 39 is an explanatory diagram for explaining a screen display according to a first modification of a ninth embodiment.

FIG. 40 is an explanatory diagram for explaining a screen display according to a second modification of the ninth embodiment.

FIG. 41 is an explanatory diagram for explaining a screen display according to a third modification of the ninth embodiment.

FIG. 42 is a flowchart for explaining a process flow of the subroutine of the area of interest extraction according to a tenth embodiment.

FIG. 43 is a functional block diagram of an information processing device according to an eleventh embodiment.

FIG. 44 is an explanatory diagram for explaining a configuration of a diagnostic support system according to a twelfth embodiment.

FIG. 45 is a functional block diagram of a server according to a thirteenth embodiment.

FIG. 46 is an explanatory diagram for explaining a configuration of a model generation system according to a fourteenth embodiment.

FIG. 47 is a functional block diagram of an information processing device according to a fifteenth embodiment.

FIG. 48 is an explanatory diagram for explaining a configuration of a diagnostic support system according to a sixteenth embodiment.

### DESCRIPTION OF EMBODIMENTS

### First Embodiment

In the present embodiment, a diagnostic support system 10 that supports a diagnosis of ulcerative colitis will be described as an example. The ulcerative colitis is one of the inflammatory bowel diseases that cause inflammation of a mucous membrane of a large intestine. It is known that the affected area occurs from a rectum to the entire circumference of a large intestine and progresses toward an entrance side.

From the fact that an active period in which symptoms appear strongly and a remission period in which symptoms disappear may be repeated and when inflammation continues, the risk of developing colorectal cancer increases, after onset, a medical follow-up by regular colonoscopy is recommended.

A doctor inserts a distal tip of a colonoscope into, for example, caecum, and then observes an endoscope image while removing the colonoscope. In the affected area, that is, the inflamed area, the inflammation is visible throughout the endoscope image.

Public institutions such as the World Health Organization (WHO), medical societies, each medical institution, and the like, respectively, establish diagnostic criteria to be used when diagnosing various diseases. For example, in the ulcerative colitis, multiple items such as a degree of reddishness of the affected area, a degree of blood vessel transparency which means the appearance of blood vessels, a degree of ulcer, and the like are listed in the diagnostic criteria.

A doctor examines each item of the diagnostic criteria, and then makes a comprehensive judgment and diagnoses an area being observed with an endoscope 14. The diagnosis includes determination of whether the area being observed is an affected area of ulcerative colitis, and determination of seriousness such as whether the area being observed is serious or light when the area being observed is an affected area. A skilled doctor will examine each item of the diagnostic criteria while removing the colonoscope to make a diagnosis of a position being observed in real time. A doctor synthesizes the diagnosis of the process of removing the colonoscope to determine the extent of the affected area inflamed by the ulcerative colitis.

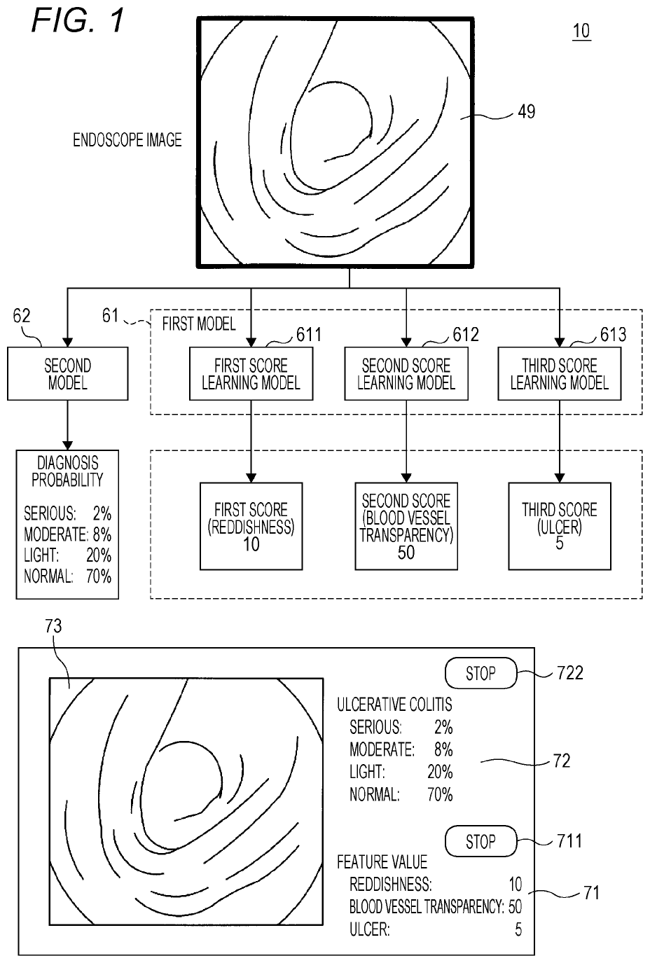

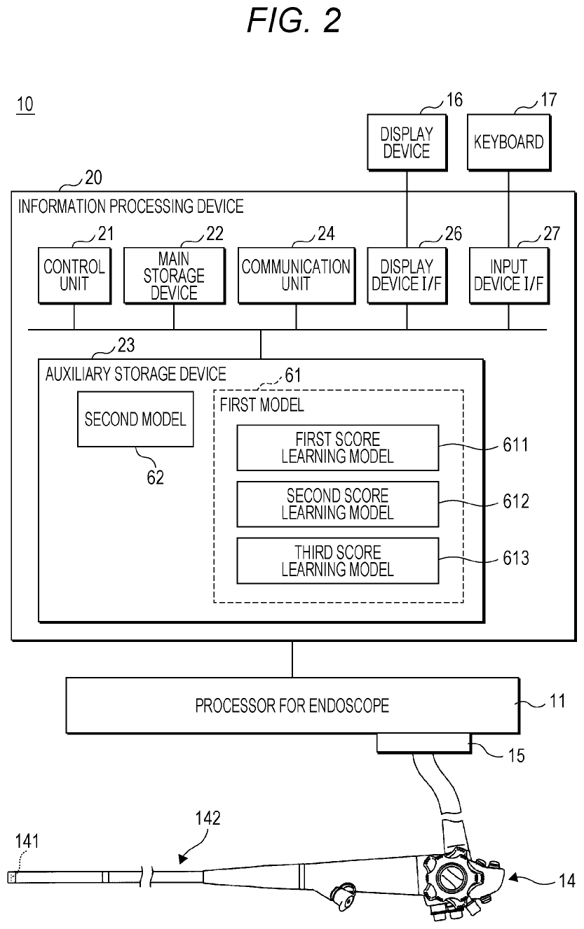

FIG. 1 is an explanatory diagram for explaining an outline of a diagnostic support system 10. An endoscope image 49 photographed using an endoscope 14 (see FIG. 2) is input to a first model 61 and a second model 62. The second model 62 outputs a diagnosis prediction regarding a state of ulcerative colitis when the endoscope image 49 is input. In the example illustrated in FIG. 1, the diagnosis prediction that the probability that the ulcerative colitis is normal, that is, the ulcerative colitis is not an affected area is 70%, and the probability that the ulcerative colitis is light is 20% is output. Details of the second model 62 will be described later.

The first model 61 includes a first score learning model 611, a second score learning model 612 and a third score learning model 613. In the following description, when there is no particular need to distinguish from the first score learning model 611 to the third score learning model 613, the first score learning model 611 to the third score learning model 613 may be simply described as the first model 61.

The first score learning model 611 outputs a predicted value of a first score obtained by digitizing evaluation regarding the degree of reddishness when the endoscope image 49 is input. The second score learning model 612 outputs a predicted value of a second score obtained by digitizing evaluation regarding the degree of blood vessel transparency when the endoscope image 49 is input. The third score learning model 613 outputs a predicted value of a third score which quantifies the evaluation regarding the degree of ulcer when the endoscope image 49 is input.

The degree of reddishness, the degree of blood vessel transparency, and the degree of ulcer are examples of diagnostic criteria items included in the diagnostic criteria used when a doctor diagnoses the condition of ulcerative colitis. The predicted values of the first to third scores are examples of diagnosis criteria prediction regarding the diagnostic criteria of ulcerative colitis.

In the example illustrated in FIG. 1, the predicted values that the first score is 10, the second score is 50, and the third score is 5 are output. Note that the first model 61 may include a score learning model that outputs a predicted value of a score obtained by digitizing evaluation regarding various diagnostic criteria items related to the ulcerative colitis, such as the degree of easy bleeding and the degree of secretion adhesion. Details of the first model 61 will be described later.

The outputs of the first model 61 and the second model 62 are acquired by a first acquisition unit and a second acquisition unit, respectively. Based on the outputs obtained by the first acquisition unit and the second acquisition unit, a screen illustrated at the bottom of FIG. 1 is displayed on a display device 16 (see FIG. 2). The screen displayed includes an endoscope image field 73, a first result field 71, a first stop button 711, a second result field 72, and a second stop button 722.

The endoscope image 49 photographed using the endoscope 14 is displayed in the endoscope image field 73 in real time. The diagnosis criteria prediction output from the first model 61 is listed in the first result field 71. The diagnosis prediction output from the second model 62 is displayed in the second result field 72.

The first stop button 711 is an example of a first reception unit that receives an operation stop instruction of the first model 61. That is, when the first stop button 711 is selected, the output of the predicted value of the score using the first model 61 is stopped. The second stop button 722 is an example of a second reception unit that receives an operation stop instruction of the second model 62. That is, when the second stop button 722 is selected, the output of the predicted value of the score using the second model 62 is stopped.

By referring to the diagnosis criteria prediction displayed in the first result field 71, a doctor checks the ground for whether the diagnosis prediction displayed in the second result field 72 is appropriate in light of the diagnostic criteria, and determines whether to adopt the diagnosis prediction displayed in the first result field 71.

FIG. 2 is an explanatory diagram for explaining a configuration of the diagnostic support system 10. The diagnostic support system 10 includes an endoscope 14, a processor 11 for endoscope, and an information processing device 20. The information processing device 20 includes a control unit 21, a main storage device 22, an auxiliary storage device 23, a communication unit 24, a display device I/F (interface) 26, an input device I/F 27, and a bus.

The endoscope 14 includes a long insertion unit 142 with an image sensor 141 provided at the distal tip thereof. The endoscope 14 is connected to the processor 11 for endoscope via an endoscope connector 15. The processor 11 for endoscope receives a video signal from the image sensor 141, performs various image processing, and generates the endoscope image 49 suitable for observation by a doctor. That is, the processor 11 for endoscope functions as an image generation unit that generates the endoscope image 49 based on the video signal acquired from the endoscope 14.

The control unit 21 is an arithmetic control device that executes the program of the present embodiment. One or more central processing units (CPUs), graphics processing units (GPUs), or multi-core CPUs, and the like are used for the control unit 21. The control unit 21 is connected to each part of hardware constituting the information processing device 20 via the bus.

The main storage device 22 is a storage device such as a static random access memory (SRAM), a dynamic random access memory (DRAM), and a flash memory. The main storage device 22 temporarily stores information required during the processing performed by the control unit 21 and a program being executed by the control unit 21.

The auxiliary storage device 23 is a storage device such as the SRAM, the flash memory, or a hard disk. The auxiliary storage device 23 stores the first model 61, the second model 62, a program to be executed by the control unit 21, and various data necessary for executing the program. As described above, the first model 61 includes the first score learning model 611, the second score learning model 612, and the third score learning model 613. Note that the first model 61 and the second model 62 may be stored in an external large-capacity storage device connected to the information processing device 20.

The communication unit 24 is an interface for data communication between the information processing device 20 and a network. The display device I/F 26 is an interface that connects the information processing device 20 and the display device 16. The display device 16 is an example of an output unit that outputs the diagnosis criteria prediction acquired from the first model 61 and the diagnosis prediction acquired from the second model 62.

The input device I/F 27 is an interface that connects the information processing device 20 and an input device such as a keyboard 17. The information processing device 20 is an information device such as a general-purpose personal computer, a tablet, or a smartphone.

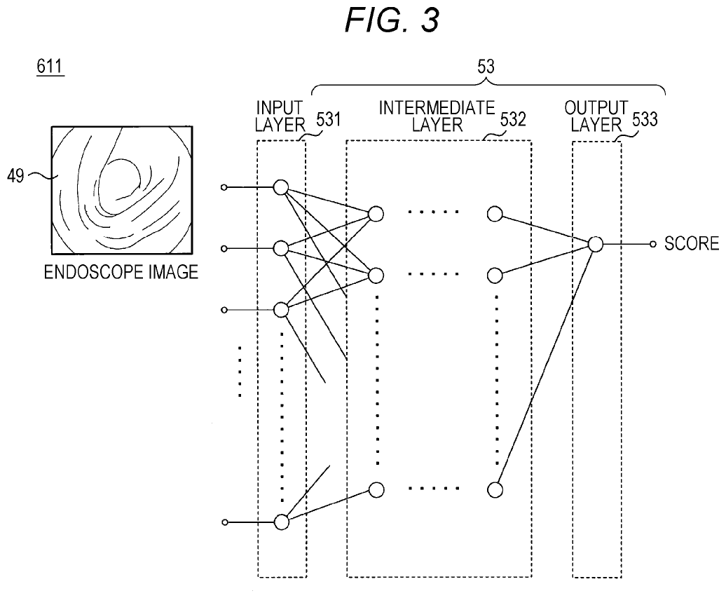

FIG. 3 is an explanatory diagram for explaining a configuration of the first score learning model 611. The first score learning model 611 outputs the predicted value of the first score when the endoscope image 49 is input.

The first score is a value obtained by digitizing, by a skilled doctor, the degree of reddishness determined based on the diagnostic criteria of ulcerative colitis when the skilled doctor looks at the endoscope image 49. For example, a doctor sets a score with a perfect score of 100 points, such as 0 points for “no reddishness” and 100 points for “strong reddishness”.

In addition, a doctor may make determination in 4 stages such as “no reddishness”, “light”, “moderate”, and “serious”, and sets a score for “no reddishness” as 0 points, a score for “light” as 1 point, a score for “moderate” as 2 points, and a score for “serious” as 3 points. The score may be set so that the “serious” becomes a smaller numerical value.

The first score learning model 611 of the present embodiment is a learning model generated by machine learning using, for example, a convolutional neural network (CNN). The first score learning model 611 includes an input layer 531, an intermediate layer 532, an output layer 533, and a neural network model 53 having a convolutional layer and a pooling layer (not illustrated). The method for generating the first score learning model 611 will be described later.

The endoscope image 49 is input to the first score learning model 611. The input image is repeatedly processed by the convolutional layer and the pooling layer, and then input to a fully-connected layer. The predicted value of the first score is output to the output layer 533.

Similarly, the second score is a numerical value obtained by determining, by a skilled specialist, the degree of blood vessel transparency based on the diagnostic criteria of ulcerative colitis when the skilled specialist looks at the endoscope image 49. Similarly, the third score is a numerical value obtained by determining, by a skilled specialist, the degree of ulcer based on the diagnostic criteria of ulcerative colitis when the skilled specialist looks at the endoscope image 49. Since the configurations of the second score learning model 612 and the third score learning model 613 are the same as those of the first score learning model 611, illustration and description thereof are omitted.

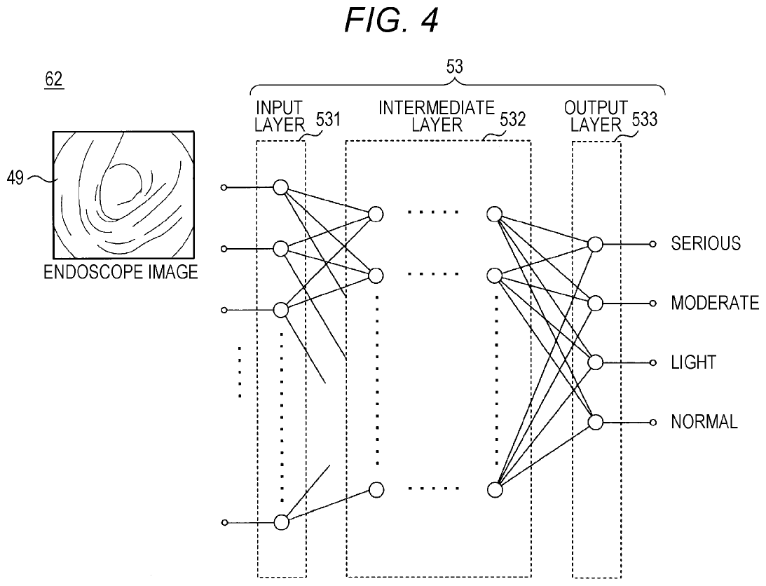

FIG. 4 is an explanatory diagram for explaining a configuration of the second model 62. The second model 62 outputs the diagnosis prediction of ulcerative colitis when the endoscope image 49 is input. The diagnosis prediction is a prediction of how a skilled specialist diagnoses the ulcerative colitis when the skilled specialist looks at the endoscope image 49.

The second model 62 of the present embodiment is a learning model generated by the machine learning using, for example, the CNN. The second model 62 includes the input layer 531, the intermediate layer 532, the output layer 533, and a neural network model 53 having the convolutional layer and the pooling layer (not illustrated). The method for generating the second model 62 will be described later.

The endoscope image 49 is input to the second model 62. The input image is repeatedly processed by the convolutional layer and the pooling layer, and then input to a fully-connected layer. The diagnosis prediction is output to the output layer 533.

In FIG. 4, the output layer 533 has four output nodes through which the probabilities that a skilled specialist determines that the ulcerative colitis is serious, the ulcerative colitis is moderate, the ulcerative colitis is light, and the ulcerative is normal, that is, the ulcerative colitis is not an affected area are output, when the skilled specialist looks at the endoscope image 49.

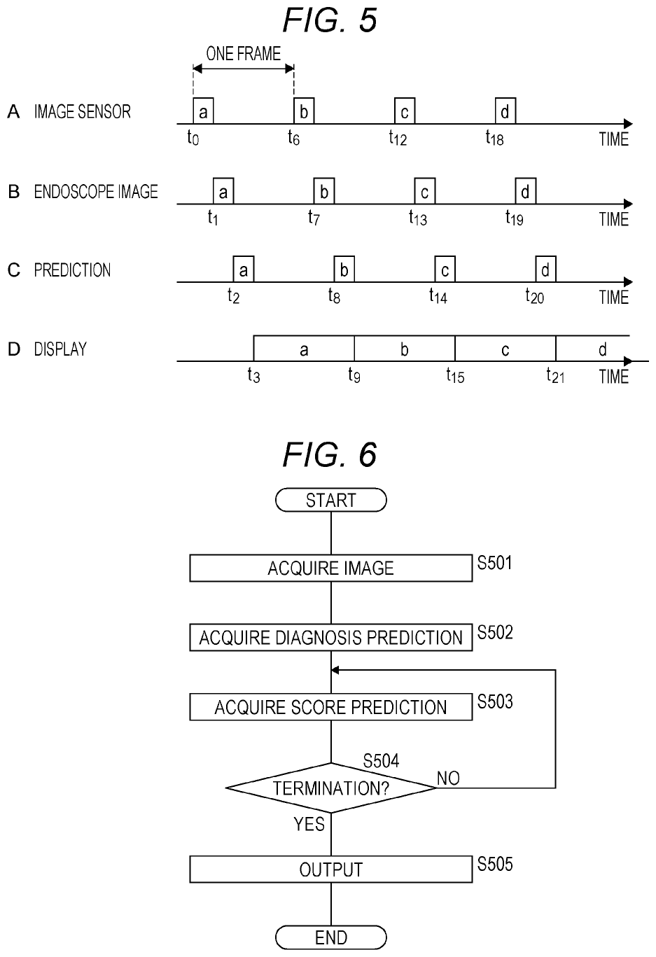

FIG. 5 is a time chart for schematically explaining an operation of the diagnostic support system 10. FIG. 5A illustrates a timing of capturing by the image sensor 141. FIG. 5B illustrates a timing of generating the endoscope image 49 by the image processing in the processor 11 for endoscope. FIG. 5C illustrates a timing when the first model 61 and the second model 62 output predictions based on the endoscope image 49. FIG. 5D illustrates a timing of display on the display device 16. All horizontal axes from FIGS. 5A to 5D indicate time.

At time t0, the image sensor 141 captures frame “a”. The video signal is transmitted to the processor 11 for endoscope. The processor 11 for endoscope performs the image processing and generates the endoscope image 49 of “a” at time t1. The control unit 21 acquires the endoscope image 49 generated by the processor 11 for endoscope and inputs the acquired endoscope image 49 to the first model 61 and the second model 62. At time t2, the control unit 21 acquires the predictions output from the first model 61 and the second model 62, respectively.

At time t3, the control unit 21 outputs the endoscope image 49 and prediction of the frame “a” to the display device 16. As a result, the processing of an image corresponding to one frame photographed by the image sensor 141 is terminated. Similarly, at time t6, the image sensor 141 captures frame “b”. At time t7, the endoscope image 49 of “b” is generated. The control unit 21 acquires the prediction at time t8, outputs the endoscope image 49 of the frame “b” at time t9, and outputs the prediction to the display device 16. Since an operation after frame “c” is also the same, a description thereof will be omitted. As a result, the endoscope image 49 and the predictions made by the first model 61 and the second model 62 are displayed in synchronization with each other.

FIG. 6 is a flowchart for explaining a process flow of a program. The program described using FIG. 6 is executed each time the control unit 21 acquires the endoscope image 49 corresponding to one frame from the processor 11 for endoscope.

The control unit 21 acquires the endoscope image 49 from the processor 11 for endoscope (step S501). The control unit 21 inputs the acquired endoscope image 49 to the second model 62, and acquires the diagnosis prediction output from the output layer 533 (step S502). The control unit 21 inputs the acquired endoscope image 49 to one of the score learning models constituting the first model 61, and acquires the predicted value of the score output from the output layer 533 (step S503).

The control unit 21 determines whether or not the process of the score learning model constituting the first model 61 is terminated (step S504). If it is determined that the process is not terminated (NO in step S504), the control unit 21 returns to step S503.

If it is determined that the process is terminated (YES in step S504), the control unit 21 generates the image described using the lower part of FIG. 1 and outputs the generated image to the display device 16 (step S505). The control unit 21 terminates the process.

According to the present embodiment, it is possible to provide the diagnostic support system 10 that displays the diagnosis criteria prediction output from the first model 61 and the diagnosis prediction output from the second model 62 together with the endoscope image 49. While observing the endoscope image 49, a doctor can check the diagnosis criteria prediction and the diagnosis prediction that predicts the diagnosis when a skilled specialist looks at the same endoscope image 49.

By referring to the diagnosis criteria prediction displayed in the first result field 71, a doctor can check the ground for whether the diagnosis prediction displayed in the second result field 72 is appropriate in light of the diagnostic criteria, and determine whether to adopt the diagnosis prediction displayed in the first result field 71.

Only the item with the highest probability and the probability may be displayed in the second result field 72. A character size can be increased by reducing the number of characters to be displayed. A doctor can detect the change in the display of the second result field 72 while gazing at the endoscope image field 73.

A doctor can stop predicting and displaying scores by selecting the first stop button 711. A doctor can stop the diagnosis prediction and the display of the diagnosis prediction by selecting the second stop button 722. A doctor can resume displaying the diagnosis prediction and diagnosis criteria prediction by reselecting the first stop button 711 or the second stop button 722.

The first stop button 711 and the second stop button 722 can be operated from any input device such as a keyboard 17, a mouse, a touch panel, or a voice input. The first stop button 711 and the second stop button 722 may be operated by using a control button or the like provided on an operation unit of the endoscope 14.

For example, when performing endoscopic pretreatment such as resection of polyps or endoscopic mucosal resection (EMR), it is preferable that a time lag from the capturing by image sensor 141 to the display on the display device 16 is as short as possible. A doctor can reduce the time lag by stopping the diagnosis prediction and the diagnosis criteria prediction by selecting the first stop button 711 and the second stop button 722.

Note that the diagnosis criteria prediction using each score learning model constituting the first model 61 and the diagnosis prediction using the second model 62 may be performed by parallel processing. By using the parallel processing, the real-time property of the display on the display device 16 can be improved.

According to the present embodiment, it is possible to provide an information processing device 20 or the like that presents a determination reason together with a determination result regarding a predetermined disease such as the ulcerative colitis. A doctor can check whether the correct result based on the diagnostic criteria is output by looking at both the diagnostic probability of disease output by the second model 62 and the score regarding the diagnostic criteria output by the first model 61.

If there is a discrepancy between the output of the second model 62 and the output of the first model 61, a doctor suspects diseases other than ulcerative colitis, consults with a medical instructor, or takes measures such as adding necessary tests. From the above, it is possible to avoid oversight of rare diseases.

The diagnosis criteria prediction using the first model 61 and the diagnosis prediction using the second model 62 may be executed by different hardware.

The endoscope image 49 may be an image recorded in an electronic medical record system or the like. For example, by inputting each image photographed at the time of the follow-up to the first model 61, it is possible to provide the diagnostic support system 10 that can compare the temporal change of each score.

### [First Modification]

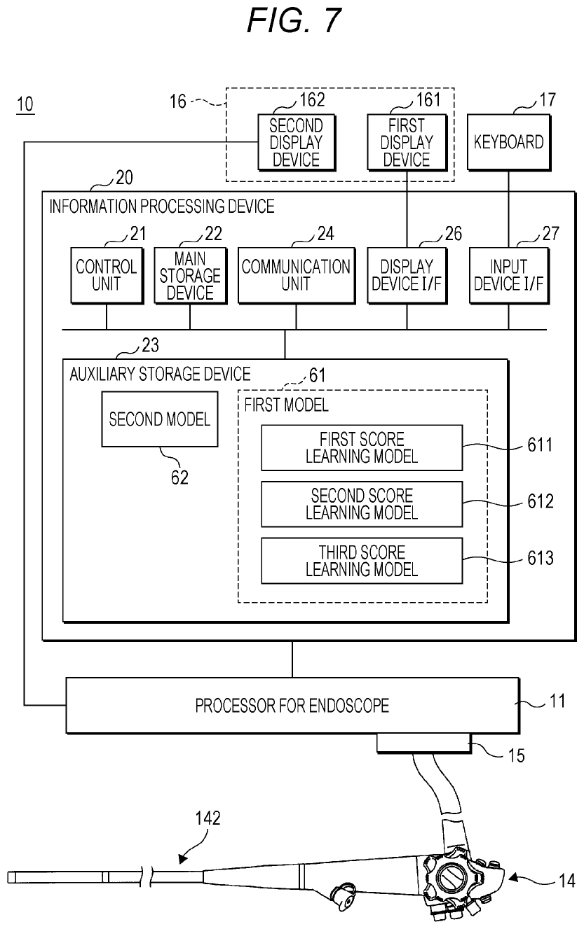

FIG. 7 is an explanatory diagram for explaining an outline of a diagnostic support system 10 according to a first modification. The description is omitted except for the differences from FIG. 2. The display device 16 includes a first display device 161 and a second display device 162. The first display device 161 is connected to a display device I/F 26. The second display device 162 is connected to a processor 11 for endoscope. It is preferable that the first display device 161 and the second display device 162 are arranged adjacent to each other.

The endoscope image 49 generated by the processor 11 for endoscope is displayed on the first display device 161 in real time. The diagnosis prediction and the diagnosis criteria prediction acquired by the control unit 21 are displayed on the second display device 162.

According to the first modification, it is possible to provide the diagnostic support system 10 that displays diagnostic prediction and diagnosis criteria prediction while reducing the display time lag of the endoscope image 49.

The diagnostic support system 10 may have three or more display devices 16. For example, the endoscope image 49 and the first result field 71 and the second result field 72 may be displayed on different display devices 16.

### [Second Modification]

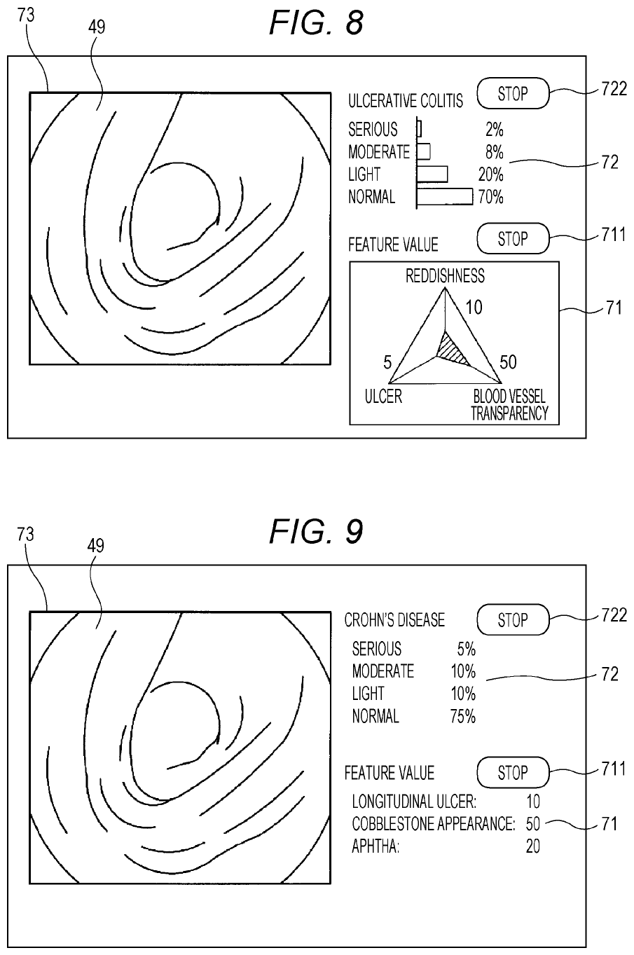

FIG. 8 is an explanatory diagram for explaining a screen display according to a second modification. The description is omitted except for the differences from the lower part of FIG. 1. In the second modification, a CPU 21 outputs the first result field 71 and the second result field 72 in graph format.

Three diagnosis criteria predictions are displayed in the first result field 71 in a three-axis graph format. In FIG. 8, an upward axis indicates a predicted value of a first score, that is, a score for reddishness. A downward right axis indicates a predicted value of a second score, that is, a score for blood vessel transparency. A downward left axis indicates a predicted value of a third score, that is, a score for ulcer.

The predicted values for the first, second, and third scores are displayed by inner triangles. In the second result field 72, the diagnosis prediction output from the second model 62 is displayed by a bar graph. According to the second modification, a doctor can intuitively grasp the diagnosis criteria prediction by looking at the triangle and bar graphs.

### [Third Modification]

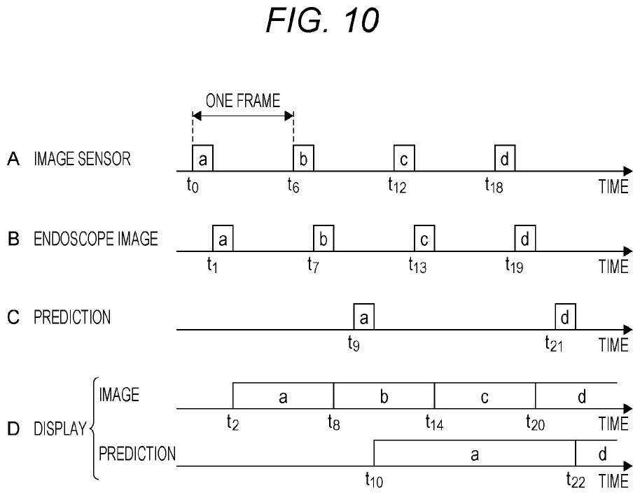

FIG. 9 is an explanatory diagram for explaining a screen display according to a third modification. FIG. 9 is a screen displayed by the diagnostic support system 10 that supports a diagnosis of Crohn's disease. Like ulcerative colitis, the Crohn's disease is a type of inflammatory bowel diseases. In FIG. 9, a first score indicates a degree of longitudinal ulcer extending in a length direction of an intestinal tract, a second score indicates a degree of cobblestone appearance that are dense mucosal ridges, and a third score is a degree of aphtha of red spots.

The diseases for which the diagnostic support system 10 supports are not limited to the ulcerative colitis and the Crohn's disease. It is possible to provide the diagnostic support system 10 that can be provided to support the diagnosis of any disease that can create the appropriate first model 61 and second model 62. It may be possible for the user to switch which disease diagnosis is assisted during the endoscope inspection. The information that assists in diagnosing each disease may be displayed on the plurality of display devices 16.

### [Fourth Modification]

FIG. 10 is a time chart for schematically explaining an operation of a fourth modification. The description of the parts common to the fifth embodiment will be omitted. FIG. 10 illustrates an example of a time chart when the processing using the first model 61 and the second model 62 takes a long time.

At time t0, the image sensor 141 captures frame “a”. The processor 11 for endoscope performs the image processing and generates the endoscope image 49 of “a” at time t1. The control unit 21 acquires the endoscope image 49 generated by the processor 11 for endoscope and inputs the acquired endoscope image 49 to the first model 61 and the second model 62. At time t2, the control unit 21 outputs an endoscope image 49 of “a” to the display device 16.

At time t6, the image sensor 141 photographs frame “b”. The processor 11 for endoscope performs the image processing and generates the endoscope image 49 of “b” at time t7. The endoscope image 49 of “b” is not input to the first model 61 and the second model 62. At time t8, the control unit 21 outputs the endoscope image 49 of “b” to the display device 16.

At time t9, the control unit 21 acquires the prediction of the endoscope image 49 of “a” output from the first model 61 and the second model 62, respectively. At time t10, the control unit 21 outputs the prediction based on the endoscope image 49 of the frame “a” to the display device 16. At time t12, the image sensor 141 photographs frame “c”. Since the subsequent process is the same as from time t0 to time t10, the description thereof will be omitted. As a result, the endoscope image 49 and the predictions made by the first model 61 and the second model 62 are displayed in synchronization with each other.

According to the fourth modification, by thinning out the endoscope image 49 input to the first model 61 and the second model 62, even if the processes using the first model 61 and the second model 62 take time, the display can be realized in real time.

### Second Embodiment

The present embodiment relates to a model generation system 19 that generates a first model 61 and a second model 62. The description of the parts common to the first embodiment will be omitted.

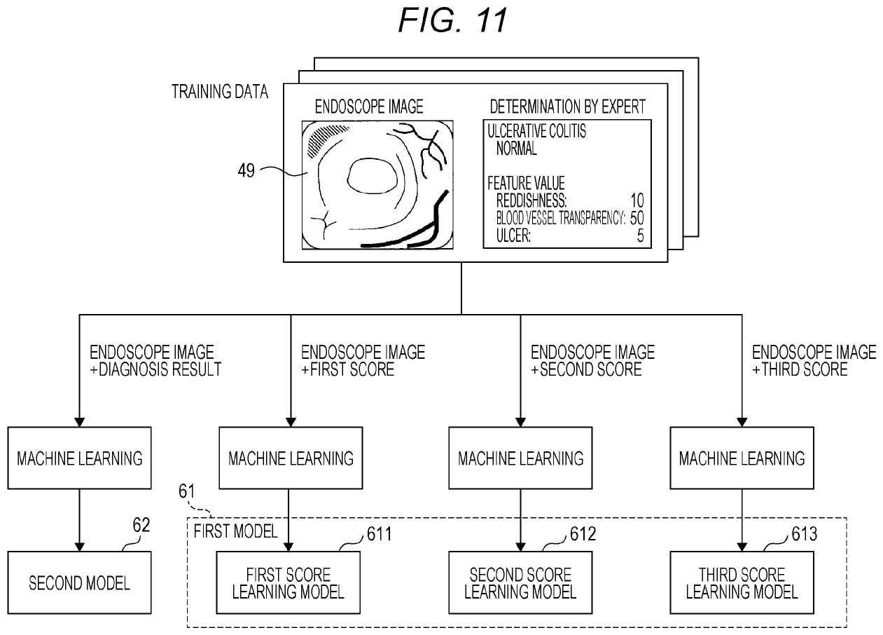

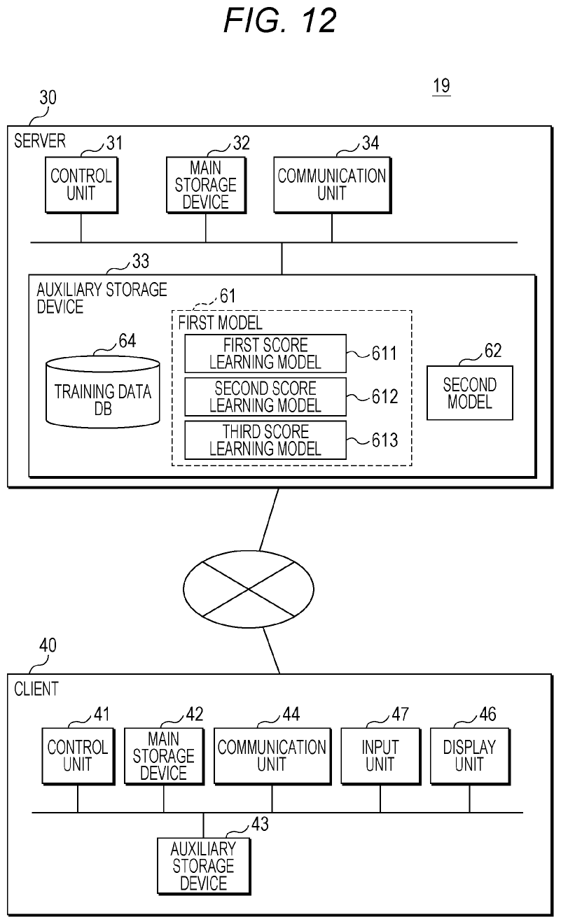

FIG. 11 is an explanatory diagram for explaining an outline of a process of generating a model. A training data DB 64 (see FIG. 12) records multiple sets of training data in which an endoscope image 49 is associated with determination results of experts such as skilled specialists. The determination results by experts are the diagnosis of ulcerative colitis, the first score, the second score, and the third score based on endoscope image 49.

A second model 62 is generated by machine learning using the set of endoscope image 49 and diagnosis result as the training data. A first score learning model 611 is generated by machine learning using the set of endoscope image 49 and first score as the training data. A second score learning model 612 is generated by machine learning using the set of endoscope image 49 and second score as the training data. A third score learning model 613 is generated by machine learning using the set of endoscope image 49 and third score as the training data.

FIG. 12 is an explanatory diagram for explaining a configuration of the model generation system 19. The model generation system 19 includes a server 30 and a client 40. The server 30 includes a control unit 31, a main storage device 32, an auxiliary storage device 33, a communication unit 34, and a bus. The client 40 includes a control unit 41, a main storage device 42, an auxiliary storage device 43, a communication unit 44, a display unit 46, an input unit 47, and a bus.

The control unit 31 is an arithmetic control device that executes the program of the present embodiment. One or more CPUs, multi-core CPUs, GPUs, or the like are used for the control unit 31. The control unit 31 is connected to each part of hardware constituting the server 30 via a bus.

The main storage device 32 is a storage device such as SRAM, DRAM, and flash memory. The main storage device 32 temporarily stores information required during the processing performed by the control unit 31 and a program being executed by the control unit 31.

The auxiliary storage device 33 is a storage device such as the SRAM, the flash memory, the hard disk, or a magnetic disk. The auxiliary storage device 33 stores the program to be executed by the control unit 31, the training data DB 64, and various data necessary for executing the program. In addition, the first model 61 and second model 62 generated by the control unit 31 are also stored in the auxiliary storage device 33. Note that the training data DB 64, the first model 61, and the second model 62 may be stored in an external large-capacity storage device or the like connected to the server 30.

The server 30 is a general-purpose personal computer, a tablet, a large computer, a virtual machine running on the large computer, a cloud computing system, or a quantum computer. The server 30 may be a plurality of personal computers or the like that perform distributed processing.

The control unit 41 is an arithmetic control device that executes the program of the present embodiment. The control unit 41 is an arithmetic control device that executes the program of the present embodiment. One or more CPUs, multi-core CPUs, GPUs, or the like are used for the control unit 41. The control unit 41 is connected to each part of hardware constituting the client 40 via the bus.

The main storage device 42 is a storage device such as SRAM, DRAM, and flash memory. The main storage device 42 temporarily stores information required during the processing performed by the control unit 41 and a program being executed by the control unit 41.

The auxiliary storage device 43 is a storage device such as the SRAM, the flash memory, or a hard disk. The auxiliary storage device 43 stores the program to be executed by the control unit 41 and various data necessary for executing the program.

The communication unit 44 is an interface for data communication between the client 40 and the network. The display unit 46 is, for example, a liquid crystal display panel, an organic electro luminescence (EL) display panel, or the like. The input unit 47 is, for example, a keyboard 17 and a mouse. The client 40 may have a touch panel in which the display unit 46 and the input unit 47 are stacked.

The client 40 is an information device such as a general-purpose personal computer, a tablet, a smartphone used by a specialist who creates training data. The client 40 may be a so-called thin client that realizes a user interface based on control by the control unit 31. When using a thin client, most of the processes performed by client 40, which will be described later, is executed by the control unit 31 instead of the control unit 41.

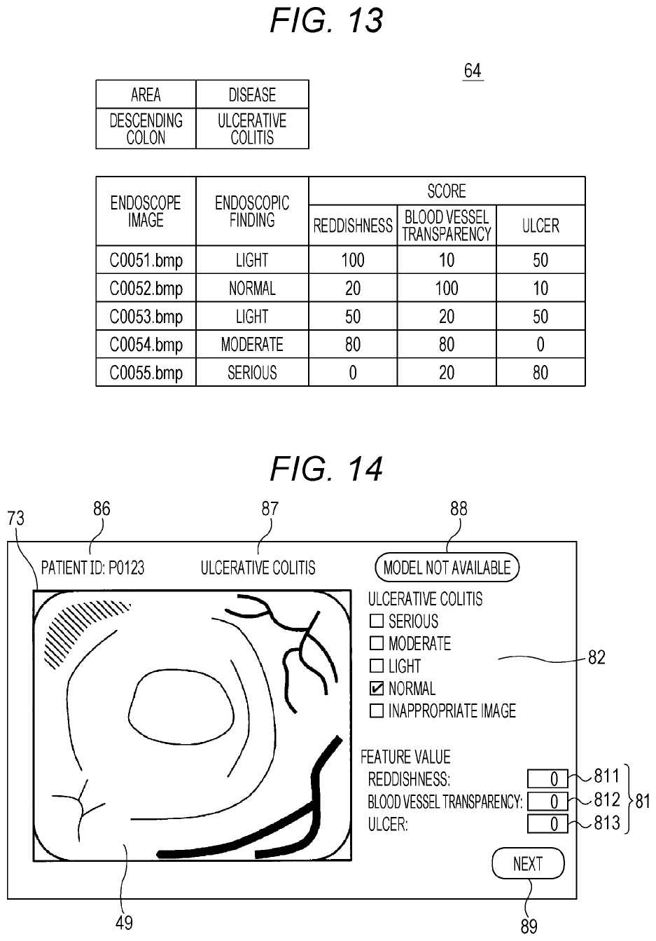

FIG. 13 is an explanatory diagram for explaining a record layout of the training data DB 64. The training data DB 64 is a DB that records the training data used to generate the first model 61 and the second model 62. The training data DB 64 has an area field, a disease field, an endoscope image field, an endoscope finding field, and a score field. The score field has a reddishness field, a blood vessel transparency field, and an ulcer field.

The site where the endoscope image 49 was photographed is recorded in the area field. A name of disease that is determined by a specialist when creating the training data is recorded in the disease field. The endoscope image 49 is recorded in the endoscope image field. The state of disease determined by a specialist or the like by looking at the endoscope image 49, that is, the endoscope finding is recorded in the endoscope finding field.

The first score regarding the reddishness, which is determined by a specialist or the like who looks at the endoscope image 49, is recorded in the reddishness field. The second score regarding the blood vessel transparency, which is determined by a specialist or the like who looks at the endoscope image 49, is recorded in the blood vessel transparency field. The third score regarding the blood vessel, which is determined by a specialist who looks at the endoscope image 49, is recorded in the ulcer field. The training data DB 64 has one record for one endoscope image 49.

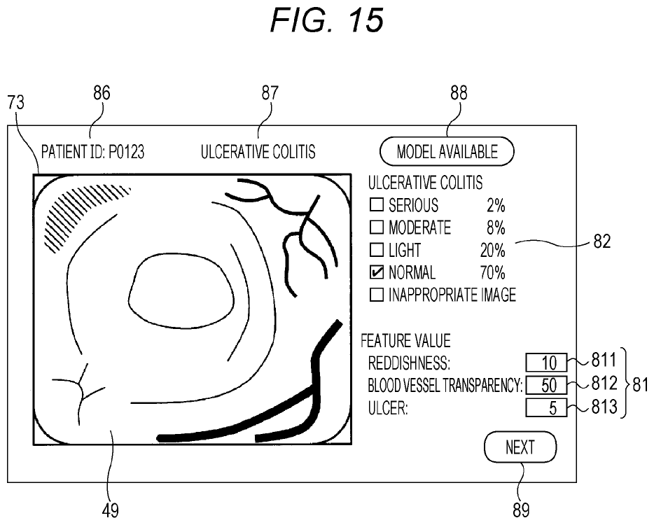

FIGS. 14 and 15 are explanatory diagrams for explaining a training data input screen. FIG. 14 illustrates an example of the screen displayed by the control unit 41 on the display unit 46 when the training data is created without using the existing first model 61 and second model 62.

The screen illustrated in FIG. 14 includes an endoscope image field 73, a first input field 81, a second input field 82, a next button 89, a patient ID field 86, a disease name field 87, and a model button 88. The first input field 81 includes a first score input field 811, a second score input field 812, and a third score input field 813. In FIG. 14, the model button 88 is set to a “model not available” state.

The endoscope image 49 is displayed in the endoscope image field 73. The endoscope image 49 may be an image photographed by the endoscope inspection performed by a specialist or the like who inputs training data, or may be an image delivered from the server 30. A specialist or the like performs a diagnosis regarding “ulcerative colitis” displayed in the disease name field 87 based on the endoscope image 49, and selects a check box provided at a left end of the second input field 82.

Note that the “inappropriate image” means that a specialist or the like determines that the endoscope image is inappropriate to use for diagnosis due to circumstances such as a large amount of residue or occurrence of blurring. The endoscope image 49 determined to be the “inappropriate image” is not recorded in the training data DB 64.

A specialist or the like determines the third score from the first score based on the endoscope image 49, and inputs the endoscope image 49 from the first score input field 811 to the third score input field 813, respectively. After the input is completed, a specialist or the like selects a next button 89. The control unit 41 transmits the endoscope image 49, the input to the first input field 81, and the input to the second input field 82 to the server 30. The control unit 31 adds a new record to the training data DB 64 to record the endoscope image 49, the endoscope finding, and each score.

FIG. 15 illustrates an example of the screen displayed by the control unit 41 on the display unit 46 when the training data is created by referring to the existing first model 61 and second model 62. In FIG. 15, the model button 88 is set to a “model available” state. Note that when the existing first model 61 and second model 62 are not generated, the model button 88 is set so that the “model available” state is not selected.

The result of inputting the endoscope image 49 to the first model 61 and the second model 62 is displayed in the first input field 81 and the second input field 82. In the second input field 82, the check box at the left end of the item with the highest probability is checked by default.

A specialist or the like determines whether each score of the first input field 81 is correct based on the endoscope image 49, and the score is changed as necessary. A specialist determines whether the check of the second input field 82 is correct based on the endoscope image 49, and reselects the check box if necessary. After the first input field 81 and the second input field 82 are in the proper state, a specialist or the like selects the next button 89. Since the subsequent processing is the same as the case of “model not available” described with reference to FIG. 14, the description thereof will be omitted.

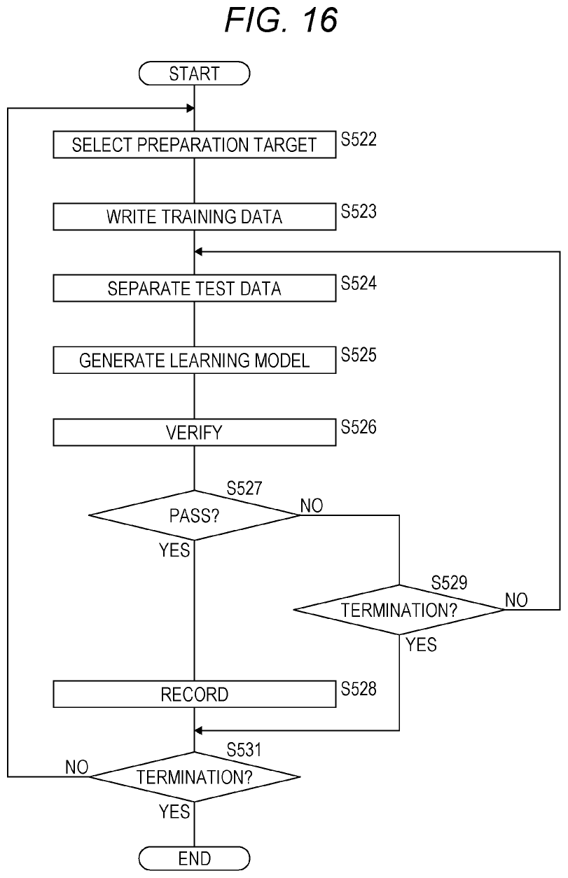

FIG. 16 is a flowchart for explaining a process flow of a program that generates a learning model. The program described with reference to FIG. 16 is used to generate each learning model that constitutes the first model 61 and the second model 62.

The control unit 31 selects the learning model to be created (step S522). The learning model to be created is any one of the learning models constituting the first model 61, or the second model 62. The control unit 31 extracts the required fields from the training data DB 64 and creates training data composed of a pair of endoscope image 49 and output data (step S523).

For example, when the first score learning model 611 is generated, the output data is the score for reddishness. The control unit 31 extracts the endoscope image field and the reddishness field from the training data DB 64. Similarly, when the second model 62 is generated, the output data is the endoscope finding. The control unit 31 extracts the endoscope image field and the endoscope finding field from the training data DB 64.

The control unit 31 separates the training data created in step S523 into training data and test data (step S524). The control unit 31 uses the training data and adjusts parameters of an intermediate layer 532 using an error back propagation method or the like to perform supervised machine learning and generate a learning model (step S525).

The control unit 31 verifies the accuracy of the learning model using the training data (step S526). The verification is performed by calculating the probability that the output matches the output data corresponding to the endoscope image 49 when the endoscope image 49 in the training data is input to the learning model.

The control unit 31 determines whether or not the accuracy of the learning model generated in step S525 is accepted (step S527). If it is determined that the accuracy of the learning model is accepted (YES in step S527), the control unit 31 records the learning model in the auxiliary storage device 33. (step S528).

If it is determined that the accuracy of the learning model is not accepted (NO in step S527), the control unit 31 determines whether to terminate the process (step S529). For example, when the processes from step S524 to step S529 are repeated a predetermined number of times, the control unit 31 determines that the process is terminated. If it is determined that the process is not terminated (NO in step S529), the control unit 31 returns to step S524.

If it is determined that the process is terminated (YES in step S529), or after the termination of step S528, the control unit 31 determines whether or not the process is terminated (step S531). If it is determined that the process is not terminated (NO in step S531), the control unit 31 returns to step S522. If it is determined that the process is terminated (YES in step S531), the control unit 31 terminates the process.

Note that when the learning model determined to be accepted is not generated, each record recorded in the training data DB 64 is reviewed and the record is added, and then the program described with reference to FIG. 16 is executed again.

The first model 61 and the second model 62 that are updated in the program described with reference to FIG. 16 are delivered to the information processing device 20 via the network or via the recording medium after the procedures such as approval under the Pharmaceutical and Medical Devices Act are completed.

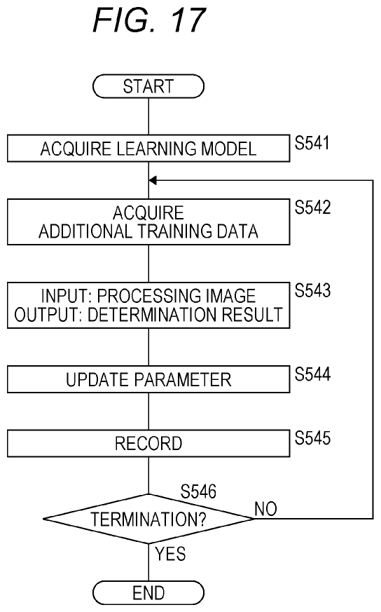

FIG. 17 is a flowchart for explaining a process flow of a program that updates a learning model. The program described with reference to FIG. 17 is executed as appropriate when additional records are recorded in the training data DB 64. Note that the additional training data may be recorded in a database different from the training data DB 64.

The control unit 31 acquires the learning model to be updated (step S541). The control unit 31 acquires additional training data (step S542). Specifically, the control unit 31 acquires the endoscope image 49 recorded in the endoscope image field and the output data corresponding to the learning model acquired in step S541 from the record added to the training data DB 64.

The control unit 31 sets the endoscope image 49 as the input data of the learning model and the output data associated with the endoscope image 49 as the output of the learning model (step S543). The control unit 31 updates the parameters of the learning model by the error back propagation method (step S544). The control unit 31 records the updated parameters (step S545).

The control unit 31 determines whether or not the process of the record added to the training data DB 64 is terminated (step S546). If it is determined that the process is not terminated (NO in step S546), the control unit 31 returns to step S542. If it is determined that the process is terminated (YES in step S546), the control unit 31 terminates the process.

The first model 61 and the second model 62 that are updated in the program described with reference to FIG. 17 are delivered to the information processing device 20 via the network or via the recording medium after the procedures such as approval under the Pharmaceutical and Medical Devices Act are completed. As a result, the first model 61 and the second model 62 are updated. Note that each learning model constituting the first model 61 and the second model 62 may be updated at the same time or individually.

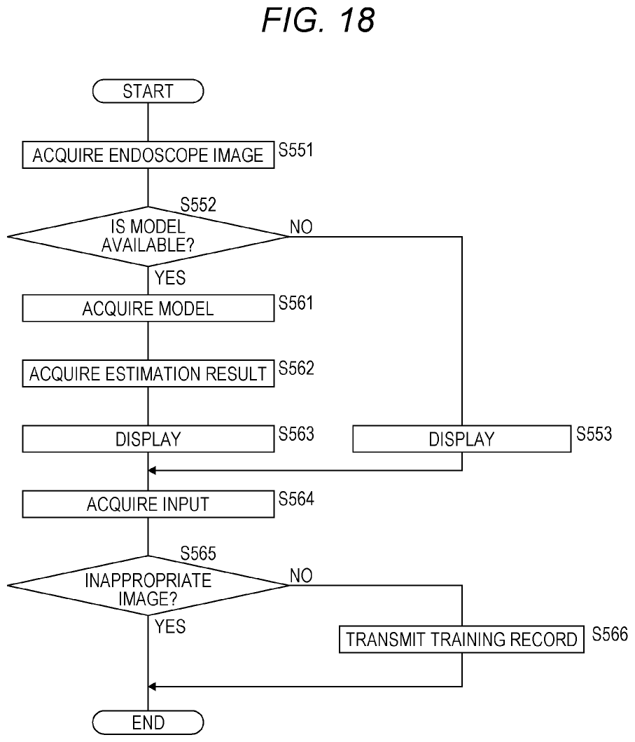

FIG. 18 is a flowchart illustrating a process flow of a program that collects the training data. The control unit 41 acquires the endoscope image 49 from an electronic medical record system (not illustrated), a hard disk mounted on the processor 11 for endoscope, or the like (step S551). The control unit 41 determines whether or not the use of the model is selected via the model button 88 described with reference to FIG. 14 (step S552).

If it is determined that the use of the model is not selected (NO in step S552), the control unit 41 displays the screen described with reference to FIG. 14 on the display unit 46 (step S553). If it is determined that the use of the model is selected (YES in step S552), the control unit 41 gets the first model 61 and the second model 62 from the server 30 (step S561).

Note that the control unit 41 may temporarily store the acquired first model 61 and second model 62 in the auxiliary storage device 43. By doing so, the control unit 41 can omit the process of the second and subsequent steps S561.

The control unit 41 inputs the endoscope image 49 acquired in step S551 to the first model 61 and the second model 62 acquired in step S561, respectively, and acquires the estimation result output from the output layer 533 (step S562). The control unit 41 displays the screen described with reference to FIG. 15 on the display unit 46 (step S563).

After the termination of step S553 or step S563, the control unit 41 acquires the input of the determination result by the user via the input unit 47 (step S564). The control unit 41 determines whether or not the “inappropriate image” is selected in the second input field 82 (step S565). If it is determined that the “inappropriate image” is selected (YES in step S565), the control unit 41 terminates the process.

If it is determined that the “inappropriate image” is not selected (NO in step S565), the control unit 41 transmits, to server 30, a training record that associates the endoscope image 49 with the input result by the user (step S566). Note that the training record may be recorded in the training data DB 64 via a portable recording medium such as a universal serial bus (USB) memory.

The control unit 31 creates a new record in training data DB 64 and records the received training record. Note that for example, when a plurality of experts make determination on the same endoscope image 49 and the determinations by a certain number of experts are matched, the endoscope image 49 may be recorded in the training data DB 64. By doing so, the accuracy of the training data DB 64 can be improved.

According to the present embodiment, the training data can be collected, and the first model 61 and second model 62 can be generated and updated.

### Third Embodiment

The present embodiment relates to a diagnostic support system 10 that outputs a score according to diagnostic criteria based on a feature quantity extracted from an intermediate layer 532 of a second model 62. The description of the parts common to the first embodiment or the second embodiment will be omitted.

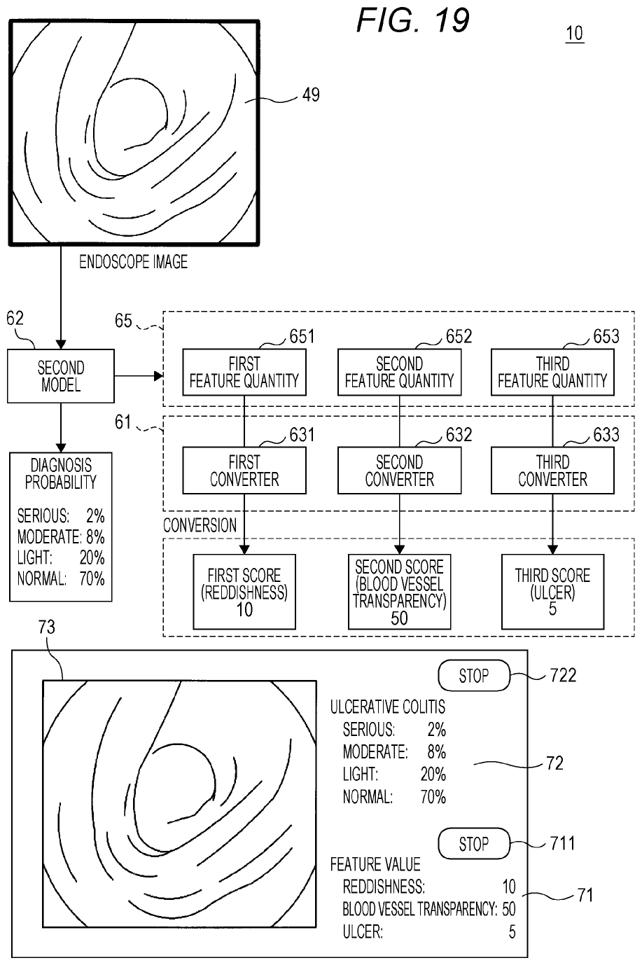

FIG. 19 is an explanatory diagram for explaining an outline of the diagnostic support system 10 according to a third embodiment. An endoscope image 49 photographed using an endoscope 14 is input to the second model 62. The second model 62 outputs the diagnosis prediction of ulcerative colitis when the endoscope image 49 is input. As will be described later, a feature quantity 65 such as a first feature quantity 651, a second feature quantity 652, and a third feature quantity 653 is acquired from nodes constituting the intermediate layer 532 of the second model 62.

The first model 61 includes a first converter 631, a second converter 632, and a third converter 633. The first feature quantity 651 is converted into a predicted value of a first score indicating a degree of reddishness by the first converter 631. The second feature quantity 652 is converted into a predicted value of a second score indicating a degree of blood vessel transparency by a second converter 632. The third feature quantity 653 is converted into a predicted value of a third score indicating a degree of ulcer by a third converter 633. When the first converter 631 to the third converter 633 are not particularly distinguished in the following description, the first converter 631 to the third converter 633 are described as a converter 63.

The outputs of the first model 61 and the second model 62 are acquired by a first acquisition unit and a second acquisition unit, respectively. Based on outputs acquired by a first acquisition unit and a second acquisition unit, a screen illustrated at the bottom of FIG. 19 is displayed on a display device 16. Since the displayed screen is the same as the screen described in the first embodiment, the description thereof will be omitted.

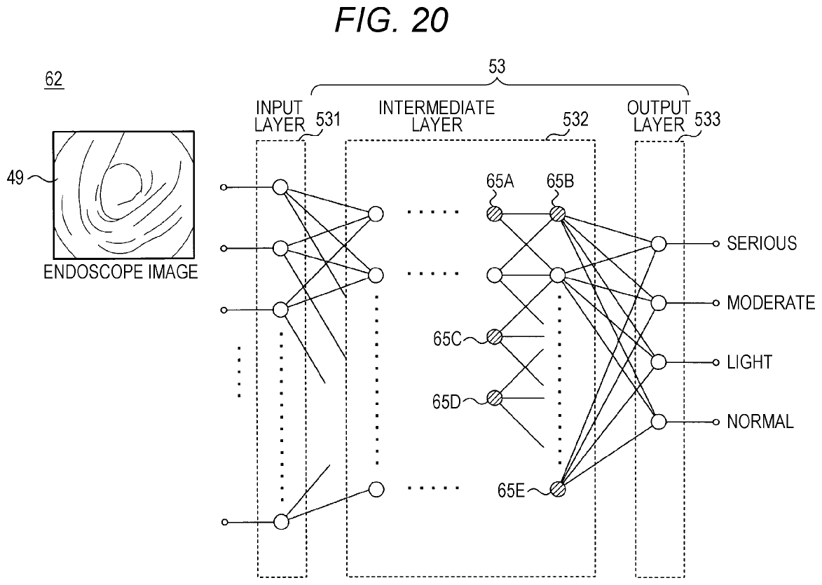

FIG. 20 is an explanatory diagram for explaining the feature quantity acquired from the second model 62. The intermediate layer 532 includes multiple nodes that are interconnected. When the endoscope image 49 is input to the second model 62, various feature quantities of endoscope image 49 appear in each node. As an example, each feature quantity that appears in five nodes is indicated by symbols from feature quantity A65A to feature quantity E65E.

The feature quantity may be acquired from a node immediately before being input to a fully-connected layer after repetitive processing is performed by a convolutional layer and a pooling layer, or may be acquired from the node included in the fully-connected layer.

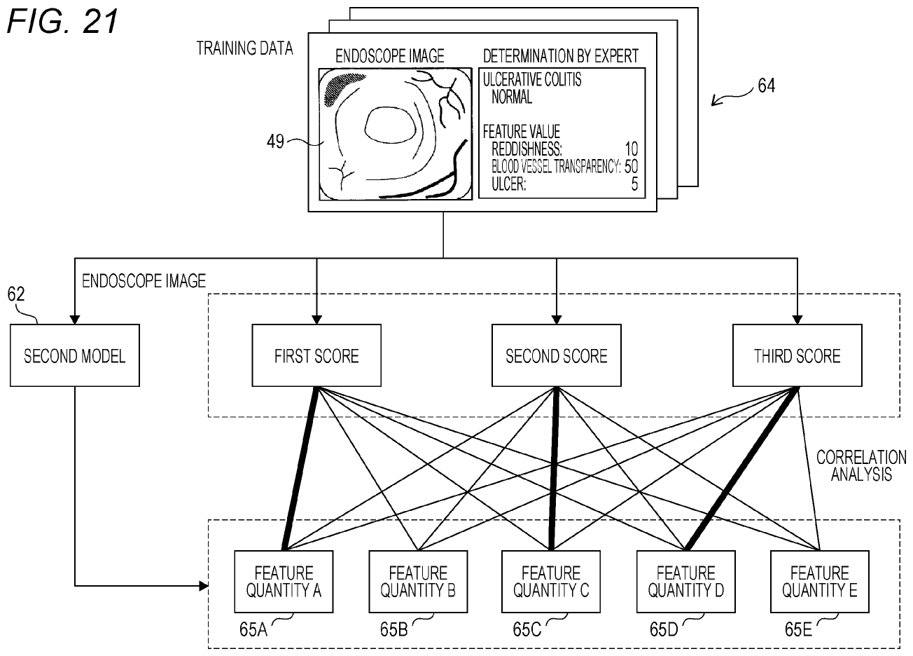

FIG. 21 is an explanatory diagram for explaining a conversion between a feature quantity and a score. The training data included in the training data DB 64 is schematically illustrated at the upper part of FIG. 21. The training data DB 64 records training data in which the endoscope image 49 is associated with the determination result by an expert such as a specialist. Since a record layout of the training data DB 64 is the same as that of the training data DB 64 of the first embodiment described with reference to FIG. 13, the description thereof will be omitted.

As described above, the endoscope image 49 is input to the second model 62, and a plurality of feature quantities such as feature quantity A65A are acquired. Correlation analysis is performed between the acquired feature quantity and the first to third scores associated with the endoscope image 49, and the feature quantity having a high correlation with each score is selected. FIG. 21 illustrates a case where a correlation between the first score and the feature quantity A65A, a correlation between the second score and feature quantity C65C, and a correlation between the third score and feature quantity D65D are high.

The first converter 631 is obtained by performing regression analysis between the first score and the feature quantity A65A. Similarly, the second converter 632 is obtained by the regression analysis of the second score and the feature quantity C65C, and the third converter 633 is obtained by the regression analysis of the third score and the feature quantity D65D, respectively. Linear regression or non-linear regression may be used for the regression analysis. The regression analysis may be performed using a neural network.

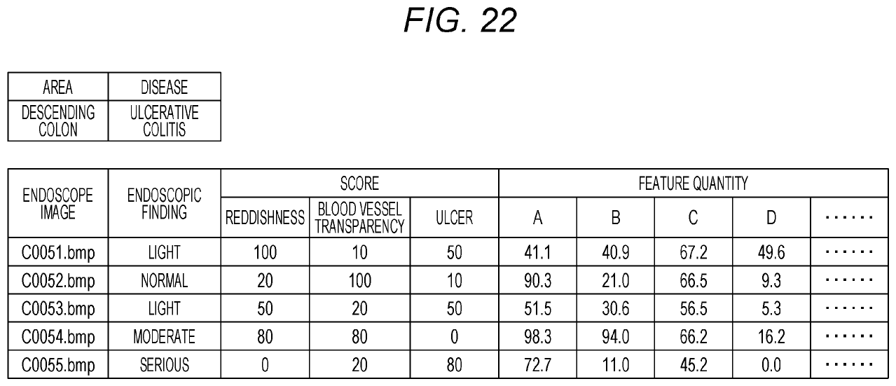

FIG. 22 is an explanatory diagram for explaining a record layout of a feature quantity DB. The feature quantity DB is a DB in which the training data and the feature quantity acquired from the endoscope image 49 are recorded in association with each other. The feature quantity DB has an area field, a disease field, an endoscope image field, an endoscope finding field, a score field, and a feature quantity field. The score field has a reddishness field, a blood vessel transparency field, and an ulcer field. The feature quantity field has a plurality of subfields such as A field and B field.

The site where the endoscope image 49 was photographed is recorded in the area field. A name of disease that is determined by a specialist when creating the training data is recorded in the disease field. The endoscope image 49 is recorded in the endoscope image field. The state of disease determined by a specialist or the like by looking at the endoscope image 49, that is, the endoscope finding is recorded in the endoscope finding field.

The first score regarding the reddishness, which is determined by a specialist or the like who looks at the endoscope image 49, is recorded in the reddishness field. The second score regarding the blood vessel transparency, which is determined by a specialist or the like who looks at the endoscope image 49, is recorded in the blood vessel transparency field. The third score regarding the ulcer, which is determined by a specialist or the like who looks at the endoscope image 49, is recorded in the ulcer field. A feature quantity such as feature quantity A64A acquired from each node of the intermediate layer 532 is recorded in each subfield of the feature quantity field.

The feature quantity DB has one record for one endoscope image 49. The feature quantity DB is stored in the auxiliary storage device 33. The feature quantity DB may be stored in an external large-capacity storage device or the like connected to the server 30.

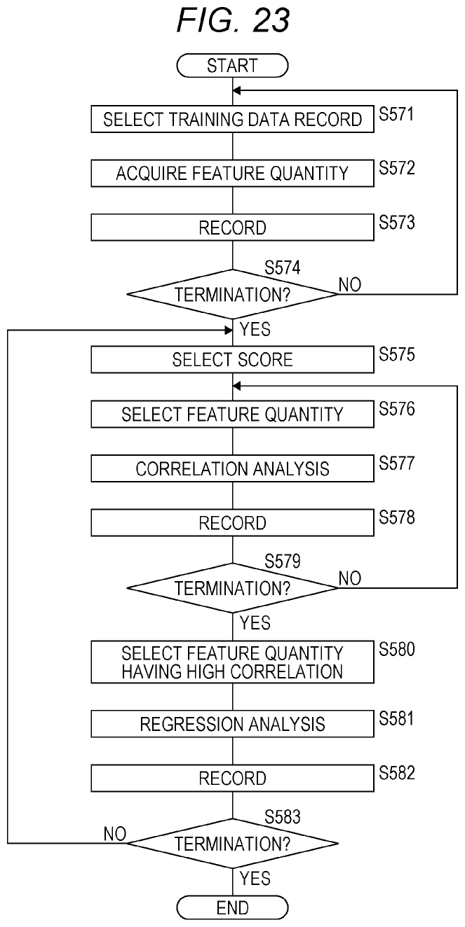

FIG. 23 is a flowchart for explaining a process flow of a program that creates the converter 63. The control unit 31 selects one record from the training data DB 64 (step S571). The control unit 31 inputs the endoscope image 49 recorded in the endoscope image field into the second model 62 and acquires the feature quantity from each node of the intermediate layer 532 (step S572). The control unit 31 creates a new record in the feature quantity DB, and records the data recorded in the record acquired in step S571 and the feature quantity acquired in step S572 (step S573).

The control unit 31 determines whether or not to terminate the process (step S574). For example, when the process of a predetermined number of training data records is terminated, the control unit 31 determines that the process is terminated. If it is determined that the process is not terminated (NO in step S574), the control unit 31 returns to step S571.

If it is determined that the process is terminated (YES in step S574), the control unit 31 selects one subfield from the score field of the feature quantity DB (step S575). The control unit 31 selects one subfield from the feature quantity field of the feature quantity DB (step S576).

The control unit 31 performs the correlation analysis between the score selected in step S575 and the feature quantity selected in step S576, and calculates the correlation coefficient (step S577). The control unit 31 temporarily records the calculated correlation coefficient in the main storage device 32 or the auxiliary storage device 33 (step S578).

The control unit 31 determines whether or not to terminate the process (step S579). For example, the control unit 31 determines that the process is terminated when the correlation analysis of all combinations of the score and the feature quantity is completed. The control unit 31 may determine that the process is terminated when the correlation coefficient calculated in step S577 is equal to or greater than a predetermined threshold.

If it is determined that the process is not terminated (NO in step S579), the control unit 31 returns to step S576. If it is determined that the process is terminated (YES in step S579), the control unit 31 selects the feature quantity that has the highest correlation with the score selected in step S575 (step S580).

The control unit 31 performs regression analysis using the score selected in step S575 as an objective variable and the feature quantity selected in step S580 as an explanatory variable, and calculates a parameter that specifies the converter 63 that converts the feature quantity into the score (step S581). For example, if the score selected in step S575 is the first score, the converter 63 specified in step S581 is the first converter 631, and if the score selected in step S575 is the second score, the converter 63 specified in step S581 is the second converter 632. The control unit 31 stores the calculated converter 63 in the auxiliary storage device 33 (step S582).

The control unit 31 determines whether or not the process of all the score fields in the feature quantity DB is terminated (step S583). If it is determined that the process is not terminated (NO in step S583), the control unit 31 returns to step S575. If it is determined that the process is terminated (YES in step S583), the control unit 31 terminates the process. As a result, each converter 63 constituting the first model 61 is generated.

The first model 61 including the converter 63 that is created in the program described with reference to FIG. 23 are delivered to the information processing device 20 via the network or via the recording medium after the procedures such as approval under the Pharmaceutical and Medical Devices Act are completed.

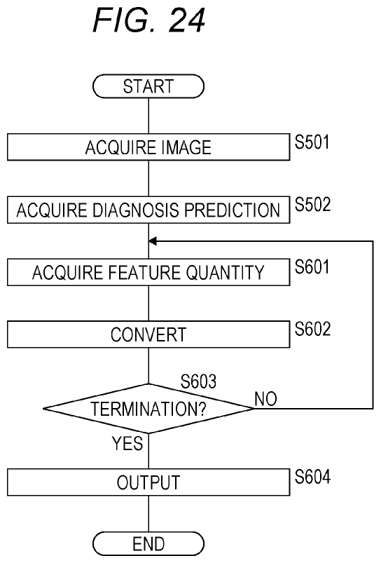

FIG. 24 is a flowchart for explaining a process flow of a program during endoscope inspection according to a third embodiment. The program in FIG. 24 is executed by the control unit 21 instead of the program described with reference to FIG. 6.

The control unit 21 acquires the endoscope image 49 from the processor 11 for endoscope (step S501). The control unit 21 inputs the acquired endoscope image 49 to the second model 62, and acquires the diagnosis prediction output from the output layer 533 (step S502).

The control unit 21 acquires the feature quantity from the predetermined node included in the intermediate layer 532 of the second model 62 (step S601). The predetermined node is a node from which the feature quantity selected in step S580 described with reference to FIG. 23 is acquired. The control unit 21 converts the acquired feature quantity by the converter 63 and calculates the score (step S602).

The control unit 21 determines whether or not all the scores are calculated (step S603). If it is determined that the process is not terminated (NO in step S603), the control unit 21 returns to step S601. If it is determined that the process is terminated (YES in step S603), the control unit 21 generates the image described with reference to the lower part of FIG. 19 and outputs the generated image to the display device 16 (step S604). The control unit 21 terminates the process.

According to the present embodiment, since the learning model generated by deep learning is only the second model 62, the diagnostic support system 10 can be realized with a relatively small amount of calculation.

By acquiring the feature quantity from the intermediate layer 532 of the second model 62, it is possible to obtain the feature quantity having a high correlation with the score without being limited to the feature quantity of the extent to which a person can normally conceive. Therefore, each diagnosis criteria prediction can be calculated accurately based on the endoscope image 49.

Note that a part of the first score, the second score, and the third score may be calculated by the same method as in the first embodiment.

### Fourth Embodiment

The present embodiment relates to an information processing system that calculates diagnosis criteria prediction based on a method other than deep learning. The description of the parts common to the first embodiment or the second embodiment will be omitted.

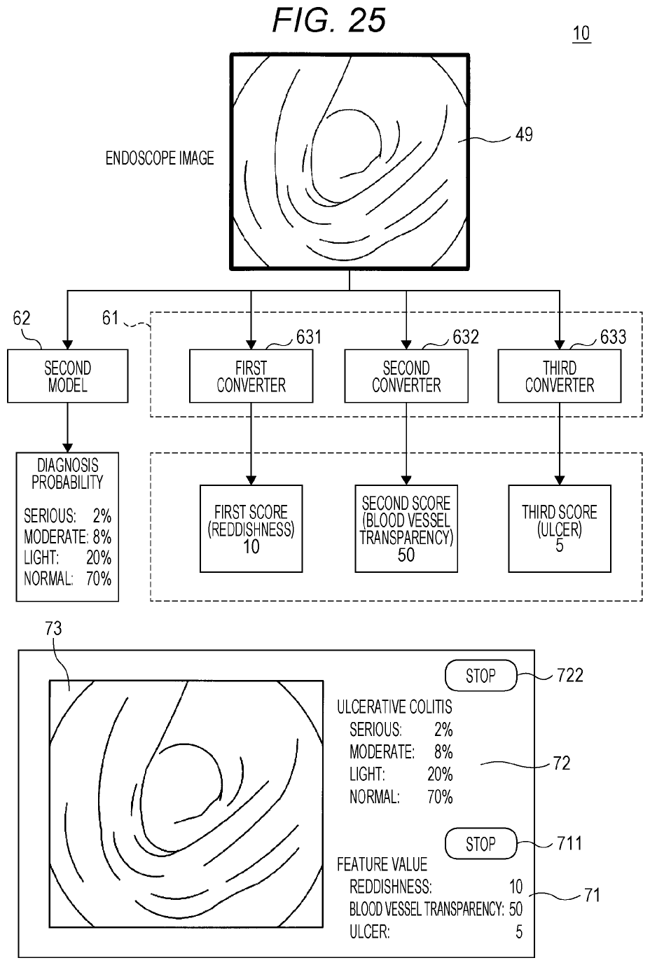

FIG. 25 is an explanatory diagram for explaining an outline of a diagnostic support system 10 according to the fourth embodiment. An endoscope image 49 photographed using an endoscope 14 is input to the second model 62. The second model 62 outputs the diagnosis prediction of ulcerative colitis when the endoscope image 49 is input.

The first model 61 includes a first converter 631, a second converter 632, and a third converter 633. The first converter 631 outputs a predicted value of a first score indicating a degree of reddishness when the endoscope image 49 is input. The second converter 632 outputs a predicted value of a second score indicating a degree of blood vessel transparency when the endoscope image 49 is input. The third converter 633 outputs a predicted value of a third score indicating a degree of ulcer when the endoscope image 49 is input.

The outputs of the first model 61 and the second model 62 are acquired by a first acquisition unit and a second acquisition unit, respectively. Based on outputs acquired by a first acquisition unit and a second acquisition unit, a screen illustrated at the bottom of FIG. 25 is displayed on a display device 16. Since the displayed screen is the same as the screen described in the first embodiment, the description thereof will be omitted.

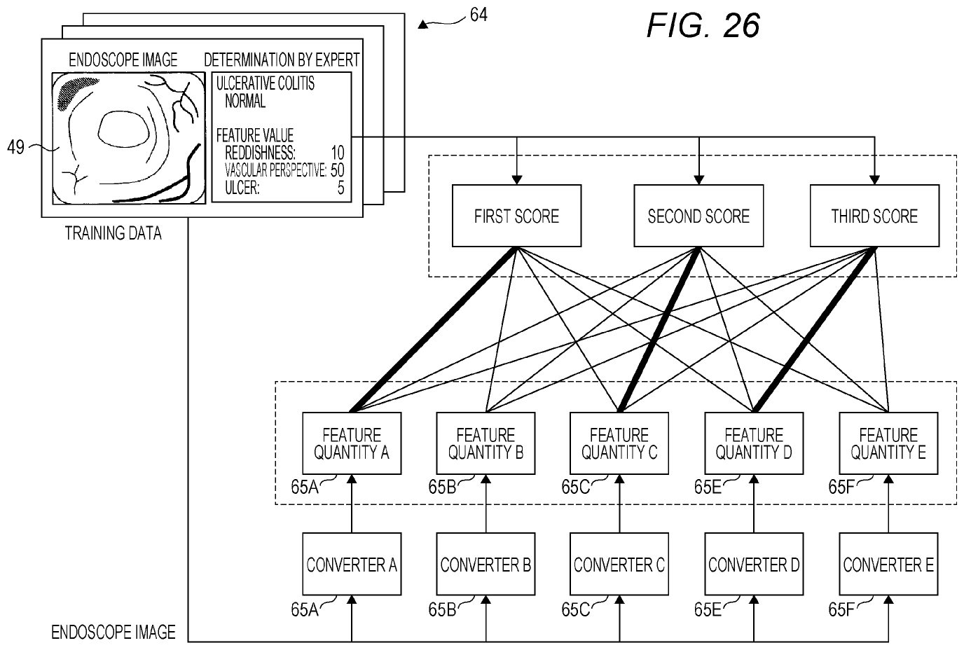

FIG. 26 is an explanatory diagram for explaining a conversion between the endoscope image 49 and the score according to the fourth embodiment. Note that in FIG. 26, the illustration of the second model 62 is omitted.

In the present embodiment, various converters 63 such as converter A63A and converter B63B that output a feature quantity when an endoscope image 49 is input are used. For example, the converter A63A converts the endoscope image 49 into the feature quantity A65A.

The converter 63 converts the endoscope image 49 into the feature quantity, for example, based on the number or ratio of pixels satisfying a predetermined condition. The converter 63 may convert the endoscope image 49 into the feature quantity by classification using a support vector machine (SVM), a random forest, or the like.

Correlation analysis is performed between the feature quantity converted by the converter 63 and the first to third scores associated with the endoscope image 49, and the feature quantity having a high correlation with each score is selected. FIG. 26 illustrates a case where the correlation between the first score and the feature quantity A65A, the correlation between the second score and the feature quantity C65C, and the correlation between the third score and the feature quantity D65D are high.

The regression analysis of the first score and the feature quantity A65A is performed, and the first converter 631 is obtained by combining with converter A63A. Similarly, the regression analysis of the first score and the feature quantity C65C is performed, and the second converter 632 is obtained by combining with converter C63C.

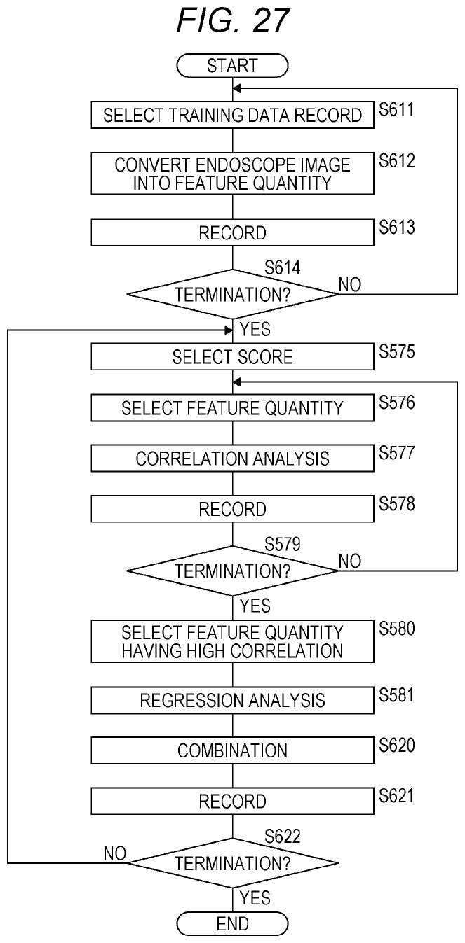

FIG. 27 is a flowchart for explaining a process flow of a program that creates the converter 63 according to the fourth embodiment. The control unit 31 selects one record from the training data DB 64 (step S611). The control unit 31 uses a plurality of converters 63 such as converter A63A and converter B63B, respectively, to convert the endoscope image 49 recorded in the endoscope image field into the feature quantity (step S612). The control unit 31 creates a new record in the feature quantity DB, and records the data recorded in the record acquired in step S611 and the feature quantity acquired in step S612 (step S613).

The control unit 31 determines whether or not to terminate the process (step S614). For example, when the process of a predetermined number of training data records is terminated, the control unit 31 determines that the process is terminated. If it is determined that the process is not terminated (NO in step S614), the control unit 31 returns to step S611.

If it is determined that the process is terminated (YES in step S614), the control unit 31 selects one subfield from the score field of the feature quantity DB (step S575). Since the processing from step S575 to step S581 is the same as the process flow of the program described with reference to FIG. 23, the description thereof will be omitted.

The control unit 31 calculates a new converter 63 by combining the result obtained by the regression analysis with the converter 63 obtained by converting the endoscope image 49 into the feature quantity in step S612 (step S620). The control unit 31 stores the calculated converter 63 in the auxiliary storage device 33 (step S621).

The control unit 31 determines whether or not the process of all the score fields in the feature quantity DB is terminated (step S622). If it is determined that the process is not terminated (NO in step S622), the control unit 31 returns to step S575. If it is determined that the process is terminated (YES in step S622), the control unit 31 terminates the process. As a result, each converter 63 constituting the first model 61 is generated.

The first model 61 including the converter 63 that is created in the program described with reference to FIG. 27 are delivered to the information processing device 20 via the network or via the recording medium after the procedures such as approval under the Pharmaceutical and Medical Devices Act are completed.

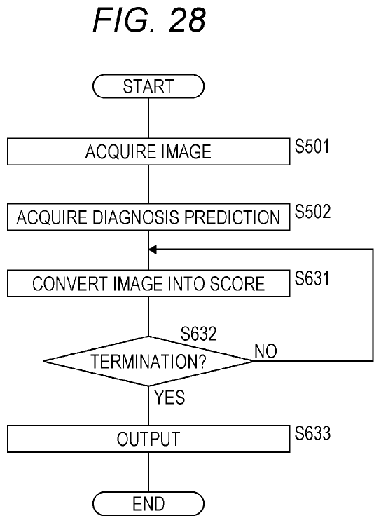

FIG. 28 is a flowchart for explaining a process flow of a program during the endoscope inspection according to the fourth embodiment. The program in FIG. 28 is executed by the control unit 21 instead of the program described with reference to FIG. 6.

The control unit 21 acquires the endoscope image 49 from the processor 11 for endoscope (step S501). The control unit 21 inputs the acquired endoscope image 49 to the second model 62, and acquires the diagnosis prediction output from the output layer 533 (step S502).

The control unit 21 inputs the acquired endoscope image 49 to the converter 63 included in the first model 61 and calculates the score (step S631).

The control unit 21 determines whether or not all the scores are calculated (step S632). If it is determined that the process is not terminated (NO in step S632), the control unit 21 returns to step S631. If it is determined that the process is terminated (YES in step S632), the control unit 21 generates the image described with reference to the lower part of FIG. 25 and outputs the generated image to the display device 16 (step S633). The control unit 21 terminates the process.

According to the present embodiment, since the learning model generated by deep learning is only the second model 62, the diagnostic support system 10 can be realized with a relatively small amount of calculation.

Note that a part of the first score, the second score, and the third score may be calculated by the same method as in the first embodiment or the third embodiment.

### Fifth Embodiment

The present embodiment relates to a diagnostic support system 10 that supports a diagnosis of diseases locally occurring such as cancer or polyps. The description of the parts common to the first embodiment or the second embodiment will be omitted.

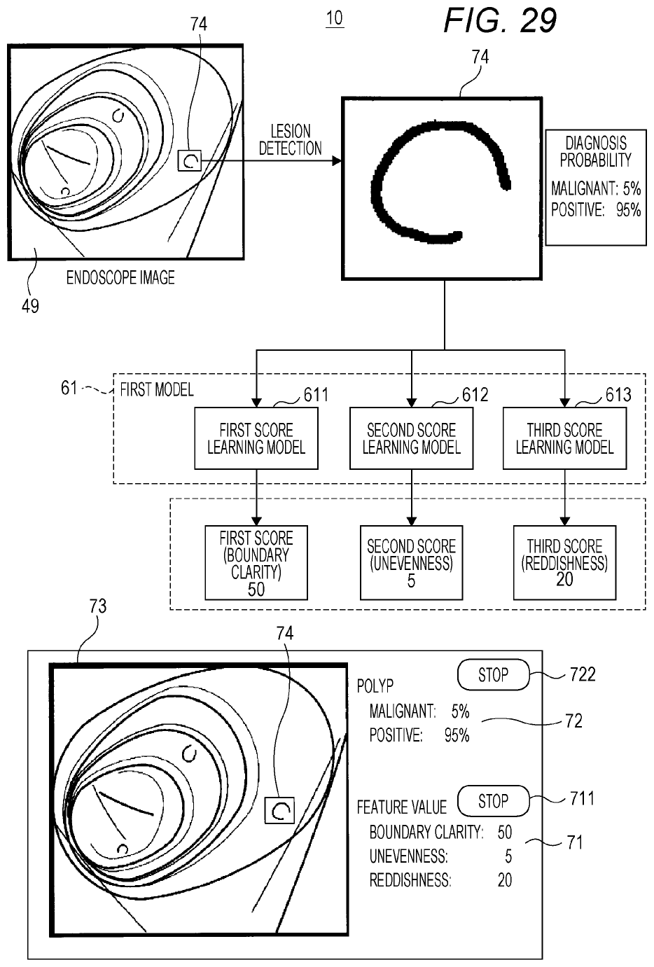

FIG. 29 is an explanatory diagram for explaining an outline of a diagnostic support system 10 according to the fifth embodiment. An endoscope image 49 photographed using an endoscope 14 is input to the second model 62. A second model 62 outputs an area prediction that predicts a range of legion region 74 that is predicted to have a lesion such as a polyp or cancer when an endoscope image 49 is input, and a diagnosis prediction such as whether the lesion is positive or malignant. In FIG. 29, it is predicted that the probability that a polyp in the legion region 74 is “malignant” is 5% and the probability that it is “positive” is 95%.

The second model 62 is a learning model that is generated using an arbitrary object detection algorithm such as regions with convolutional neural network (RCNN), fast RCNN, faster RCNN, single shot multibook detector (SSD), or You Only Look Once (YOLO). Since the learning model that accepts the input of the medical image and outputs the region where the lesion exists and the diagnosis prediction is output is conventionally used, the detailed description thereof will be omitted.

The first model 61 includes a first score learning model 611, a second score learning model 612 and a third score learning model 613. The first score learning model 611 outputs the predicted value of the first score, which indicates the degree of boundary clarity, when the image in the legion region 74 is input. The second score learning model 612 outputs the predicted value of the second score indicating the degree of unevenness of a surface when the image in the legion region 74 is input. The third score learning model 613 outputs the predicted value of the third score indicating the degree of reddishness when the image in the legion region 74 is input.

In the example illustrated in FIG. 29, the predicted values that the first score is 50, the second score is 5, and the third score is 20 are output. Note that the first model 61 may include a score learning model that outputs diagnosis criteria predictions related to various diagnostic criteria items related to polyps, such as a shape of pedunculated or not, and the degree of secretion adhesion.

The outputs of the first model 61 and the second model 62 are acquired by a first acquisition unit and a second acquisition unit, respectively. Based on outputs acquired by a first acquisition unit and a second acquisition unit, a screen illustrated at the bottom of FIG. 29 is displayed on a display device 16. Since the displayed screen is the same as the screen described in the first embodiment, the description thereof will be omitted.

If multiple legion regions 74 are detected in the endoscope image 49, each legion region 74 is input to the first model 61 and a diagnosis criteria prediction is output. The user can view the diagnosis prediction and the score related to the legion region 74 by selecting the legion region 74 displayed in the endoscope image field 73. Note that the diagnosis predictions and scores for a plurality of legion regions 74 may be listed on the screen.

The legion region 74 may be surrounded by a circle, an ellipse, or any closed curve. In such a case, the peripheral area is masked with black or white, and thus the image corrected to a shape suitable for input to the first model 61 is input to the first model 61. For example, when multiple polyps are close to each other, the region including one polyp can be cut out and the score can be calculated by the first model 61.

### Sixth Embodiment

The present embodiment relates to a diagnostic support system 10 that outputs the probability that a first model 61 is in each category defined in diagnostic criteria for diseases. The description of the parts common to the first embodiment will be omitted.

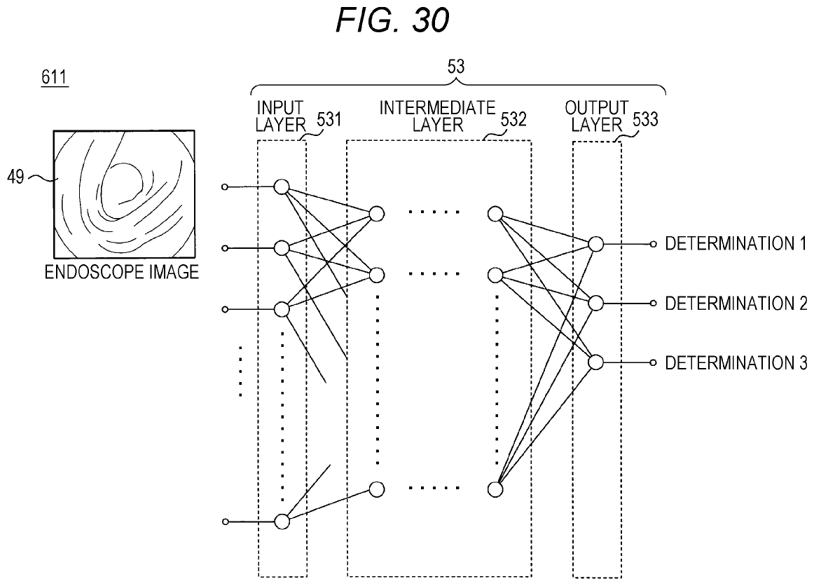

FIG. 30 is an explanatory diagram for explaining a configuration of a first score learning model 611 according to the sixth embodiment. The first score learning model 611 described with reference to FIG. 30 is used in place of the first score learning model 611 described with reference to FIG. 3.

In the first score learning model 611, when an endoscope image 49 is input, an output layer 533 has three output nodes that output the probability that the degree of reddishness is each of the three stages of “determination 1”, “determination 2”, and “determination 3” based on the diagnostic criteria of ulcerative colitis. The “determination 1” means that the degree of reddishness is “normal”, the “determination 2” means “erythema”, and the “determination 3” means “strong erythema”.

Similarly, in the second score learning model 612, the “determination 1” means that the degree of blood vessel transparency is “normal”, the “determination 2” means that the blood vessel transparency is “disappearance into erythema”, and the “determination 3” means that the blood vessel transparency is “disappearance” throughout almost the entire area.

Note that the number of nodes in the output layer 533 of the score learning model is arbitrary. In the present embodiment, the third score learning model 613 has four output nodes from the “determination 1” to the “determination 4” in the output layer 533. The “determination 1” means that the degree of ulcer is “none”, the “determination 2” means that the degree of ulcer is “erosion”, the “determination 3” means that the degree of ulcer is “medium” depth ulcer, and the “determination 4” means that the degree of ulcer is “deep” ulcer, respectively.

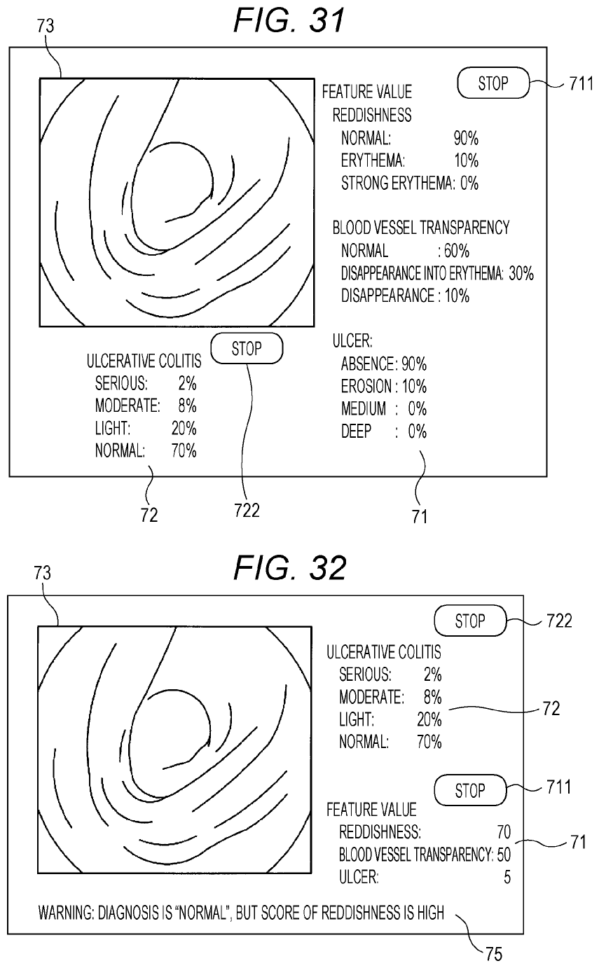

FIG. 31 is an explanatory diagram for explaining a screen display according to the sixth embodiment. An endoscope image field 73 is displayed in the upper left of the screen. A first result field 71 and a first stop button 711 are displayed on the right side of the screen. A second result field 72 and a second stop button 722 are displayed under the endoscope image field 73.

According to the present embodiment, it is possible to provide the diagnostic support system 10 that displays the first result field 71 in an expression according to the definition defined in the diagnostic criteria.

### Seventh Embodiment

The present embodiment relates to a diagnostic support system 10 that displays a warning when there is a discrepancy between an output by a first model 61 and an output by a second model 62. The description of the parts common to the first embodiment will be omitted.

FIG. 32 is an explanatory diagram for explaining a screen display according to the seventh embodiment. In the example illustrated in FIG. 32, the diagnosis criteria prediction that the probability of being normal is 70%, a first score indicating a degree of reddishness is 70, a second score indicating a degree of blood vessel transparency is 50, and a third score indicating a degree of ulcer is 5, is output.

A warning field 75 is displayed at the bottom of the screen. The warning field 75 should be judged as not “normal” when the first score, which is the degree of “reddishness” according to diagnostic criteria, is high, and thus indicates that there is a discrepancy between first result field 71 and second result field 72. The presence or absence of the discrepancy is determined on a rule basis based on the diagnostic criteria.

In this way, when there is the discrepancy between the output by the first model 61 and the output by the second model 62, the warning field 75 is displayed to call the attention of the doctor that is the user.

### Eighth Embodiment

The present embodiment relates to a diagnostic support system 10 in which a processor 11 for endoscope and an information processing device 20 are integrated. The description of the parts common to the first embodiment will be omitted.

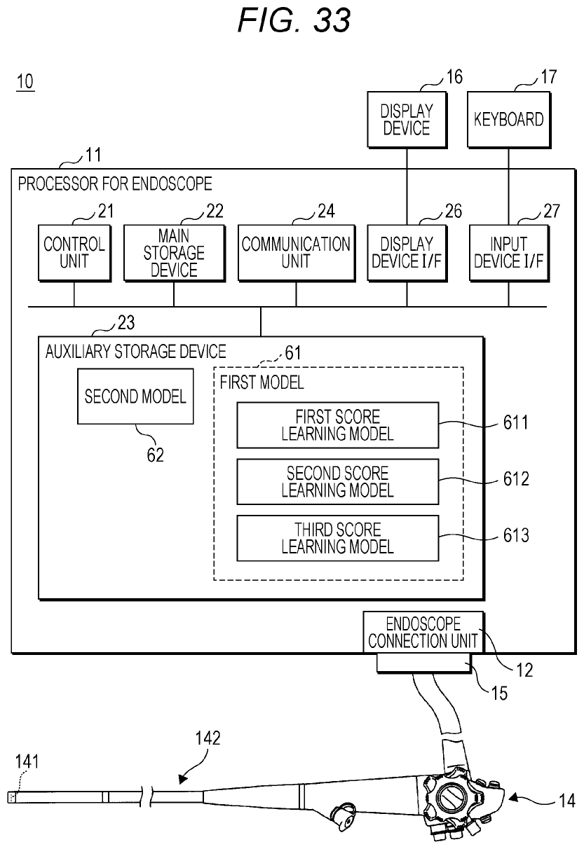

FIG. 33 is an explanatory diagram for explaining an outline of the diagnostic support system 10 according to the eighth embodiment. Note that in FIG. 33, illustration and description of a configuration for realizing basic functions of the processor 11 for endoscope, such as a light source, an air supply/water supply pump, and a control unit of an image sensor 141, will be omitted.

The diagnostic support system 10 includes an endoscope 14 and the processor 11 for endoscope. The processor 11 for endoscope includes an endoscope connection unit 12, a control unit 21, a main storage device 22, an auxiliary storage device 23, a communication unit 24, a display device I/F 26, an input device I/F 27, and a bus.

Since the control unit 21, the main storage device 22, the auxiliary storage device 23, the communication unit 24, the display device I/F 26, and the input device I/F 27 are the same as those in the first embodiment, the description thereof will be omitted. The endoscope 14 is connected to the endoscope connection unit 12 for endoscope via the endoscope connector 15.

According to the present embodiment, the control unit 21 receives a video signal from the endoscope 14 via the endoscope connection unit 12 and performs various image processing to generate the endoscope image 49 suitable for observation by a doctor. The control unit 21 inputs the generated endoscope image 49 to a first model 61 and acquires the diagnosis criteria prediction of each item according to the diagnostic criteria. The control unit 21 inputs the generated endoscope image 49 to the second model 62 and acquires the diagnosis prediction of the disease.

Note that the first model 61 and the second model 62 may be configured to accept the video signal acquired from the endoscope 14 or an image in the process of generating the endoscope image 49 based on the video signal. By doing so, it is possible to provide the diagnostic support system 10 that can also use information lost in the process of generating an image suitable for observation by a doctor.

### Ninth Embodiment

The present embodiment relates to a diagnostic support system 10 that displays an area in the endoscope image 49 that affects diagnosis criteria prediction output from a first model 61. The description of the parts common to the first embodiment will be omitted.

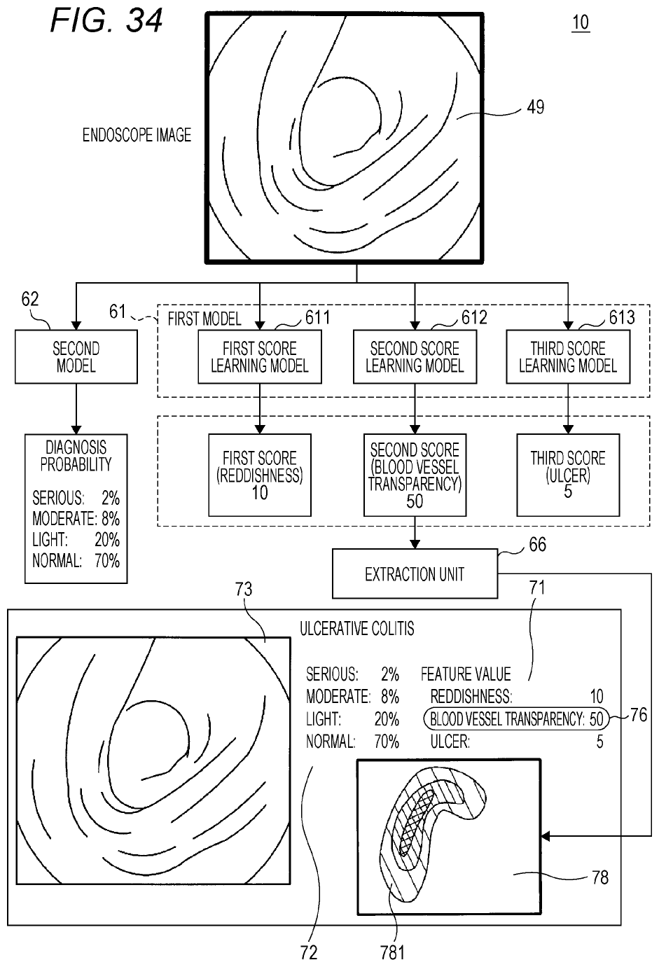

FIG. 34 is an explanatory diagram for explaining an outline of the diagnostic support system 10 according to the ninth embodiment. FIG. 34 illustrates the diagnostic support system 10 in which an extraction unit 66 for extracting the area affecting the second score is added to the diagnostic support system 10 of the first embodiment described with reference to FIG. 1.

As in the first embodiment, the endoscope image 49 is input to the first model 61 and the second model 62, and each output is acquired by the first acquisition unit and the second acquisition unit. Of the endoscope image 49, the area of interest that affects the second score is extracted by extraction unit 66.

The extraction unit 66 can be realized by an algorithm of a known area of interest visualization method such as class activation mapping (CAM), gradient-weighted class activation mapping (Grad-CAM), or Grad-CAM++.

The extraction unit 66 may be realized by software executed by the control unit 21 or by hardware such as an image processing chip. In the following description, the case where the extraction unit 66 is realized by software will be described as an example.

The control unit 21 displays the screen shown at the bottom of FIG. 34 on the display device 16 based on the output acquired by the first acquisition unit and the second acquisition unit and the area of interest extracted by the extraction unit 66. The screen displayed includes an endoscope image field 73, a first result field 71, a second result field 72, and an area of interest field 78.

The endoscope image 49 photographed using the endoscope 14 is displayed in the endoscope image field 73 in real time. The diagnosis criteria prediction output from the first model 61 is listed in the first result field 71. The diagnosis prediction output from the second model 62 is displayed in the second result field 72.

In the example illustrated in FIG. 34, a select cursor 76 indicates that a user selects the item of the term “blood vessel transparency”, which indicates the second score of the first result field 71.

In the area of interest field 78, the area of interest extracted by the extraction unit 66 is displayed by an area of interest indicator 781. The area of interest indicator 781 expresses the magnitude of the influence on the second score by a heat map or a contour line display. In FIG. 34, an area of interest indicator 781 may be represented by a frame surrounding an area having a stronger influence on a second score than a predetermined threshold, where the area of interest indicator 781 is displayed by using detailed hatching as the influence on the diagnosis criteria prediction is stronger than the predetermined threshold.

Note that when the item of the “reddishness” indicating the first score is selected by the user, the select cursor 76 is displayed in the item of the “reddishness”. The extraction unit 66 extracts the area that affects the first score. Similarly, when the item of the “ulcer” indicating the third score is selected by the user, the select cursor 76 is displayed in the item of the “ulcer”. The extraction unit 66 extracts the area that affects the third score. If none of the diagnostic criteria items are selected by the user, the select cursor 76 is not displayed and the area of interest indicator 781 is not displayed in an area of interest field 78.

The diagnostic support system 10 may be able to receive the selection of a plurality of diagnostic criteria items at the same time. By doing so, the diagnostic support system 10 has multiple extraction units 66 that extract the area that affected the diagnosis criteria prediction for each diagnostic criteria item that accepts the selection.

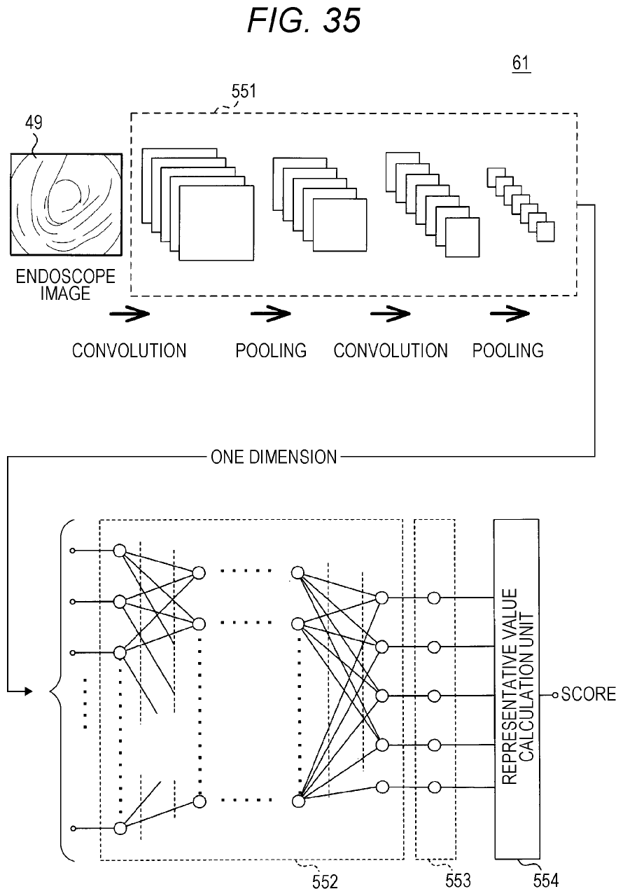

FIG. 35 is an explanatory diagram for explaining a configuration of the first model 61. In the present embodiment, the configuration of the first model 61, which is schematically described with reference to FIG. 3, will be described in more detail.

The endoscope image 49 is input to a feature quantity extraction unit 551. The feature quantity extraction unit 551 is constituted by a convolutional layer and a pooling layer that are repeated. In the convolutional layer, the convolution processing is performed between each of the plurality of filters and the input image. In FIG. 35, a stacked quadrangle schematically show an image that has undergone convolution processing with different filters.

In the pooling layer, the input image is reduced. In the final layer of the feature quantity extraction unit 551, multiple small images that reflect various features of the original endoscope image 49 are generated. Data in which each pixel of these images is arranged in one dimension is input to a fully-connected layer 552. The parameters of the feature quantity extraction unit 551 and the fully-connected layer 552 are adjusted by machine learning.

The output of the fully-connected layer 552 is adjusted by the soft mask layer 553 so that the total is 1, and the prediction probability of each node is output from the soft mask layer 553. Table 1 shows an example of the output of the soft mask layer 553.

TABLE 1

| Output node number | Range of score |
|---|---|
| 1 | 0 or more and less than 10 |
| 2 | 20 or more and less than 40 |
| 3 | 40 or more and less than 60 |
| 4 | 60 or more and less than 80 |
| 5 | 80 or more and 100 or less |

For example, a first node of the soft mask layer 553 outputs the probability that the value of the first score is 0 or more and less than 20. The probability that the value of the first score is 20 or more and less than 40 is output from a second node of the soft mask layer 553. The sum of the probabilities of all nodes is 1.

A representative value calculation unit 554 calculates the score, which is the representative value of the output of the soft mask layer 553, and outputs the score. The representative value is, for example, an expected value, a median value, or the like of the score.

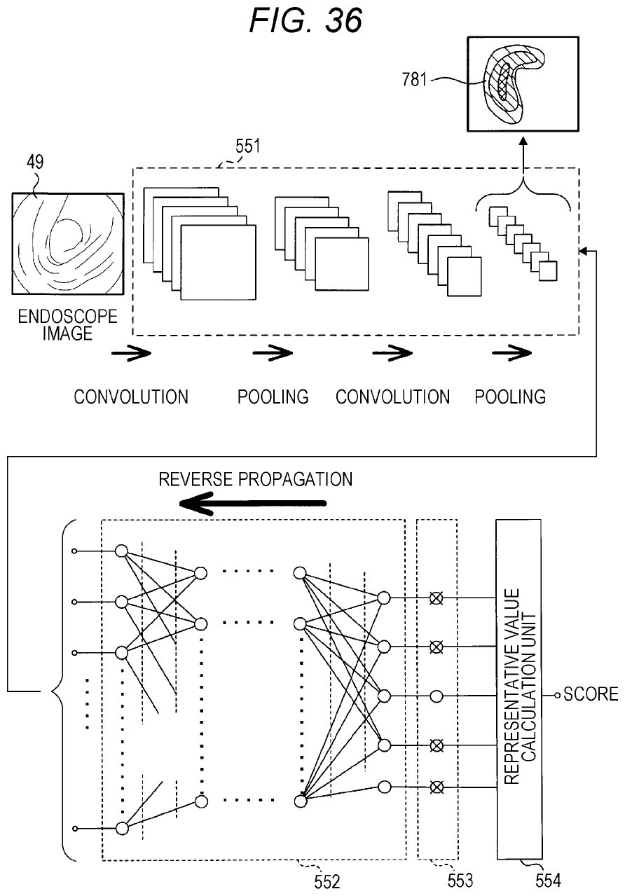

FIG. 36 is an explanatory diagram for explaining a configuration of the extraction unit 66. The control unit 21 sets “1” for the output node of the soft mask layer 553 corresponding to the score calculated by the representative value calculation unit 554, and “0” for the other output nodes. The control unit 21 calculates the back propagation of the fully-connected layer 552.

The control unit 21 generates a heat map based on an image of a final layer of the feature quantity extraction unit 551 obtained by the back propagation. As a result, the area of interest indicator 781 is defined.

The heat map can be generated by the known methods such as class activation mapping (CAM), gradient-weighted class activation mapping (Grad-CAM), or Grad-CAM++.

Note that the control unit 21 may perform the back propagation of the feature quantity extraction unit 551 and generate the heat map based on the image other than the final layer.

For example, when using the Grad-CAM, specifically, the control unit 21 accepts a model type of first score learning model 611, second score learning model 612, or third score learning model 613, or a name of any of multiple convolutional layers. The control unit 21 inputs the accepted model type and layer name in the Grad-CAM code, finds the gradient, and generates the heat map. The control unit 21 displays the generated heat map and the model name and layer name corresponding to the heat map on the display device 16.

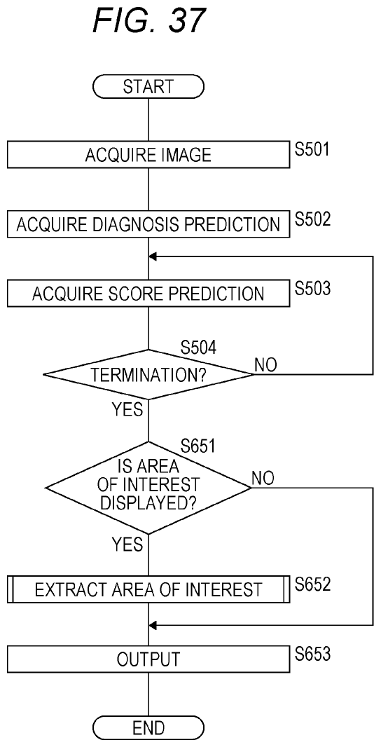

FIG. 37 is a flowchart for explaining a process flow of a program according to the ninth embodiment. The program in FIG. 37 is executed by control unit 21 instead of the program described with reference to FIG. 6. Since the processes from step S501 to step S504 is the same as the process flow of the program described with reference to FIG. 6, the description thereof will be omitted.

The control unit 21 determines whether or not the display selection regarding the area of interest is accepted (step S651). If it is determined that the selection is accepted (YES in step S651), the control unit 21 executes an area of interest extraction subroutine (step S652). The area of interest extraction subroutine is a subroutine that extracts an area of interest that affects a predetermined diagnosis criteria prediction from the endoscope image 49. The process flow of the area of interest extraction subroutine will be described later.

If it is determined that the selection is not accepted (NO in step S651), or after the end of step S652, the control unit 21 generates an image described with reference to the lower part of FIG. 34 and outputs the generated image to display device 16 (step S653). After that, the control unit 21 terminates the process.

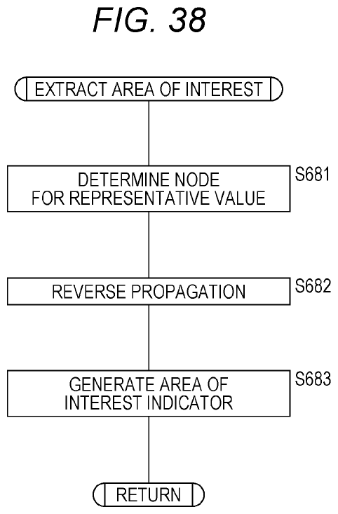

FIG. 38 is a flowchart for explaining a process flow of the area of interest extraction subroutine. The area of interest extraction subroutine is a subroutine that extracts an area of interest that affects a predetermined diagnosis criteria prediction from the endoscope image 49. The area of interest extraction subroutine realizes the function of extraction unit 66 by software.

The control unit 21 determines the output node of the soft mask layer 553, which corresponds to the score calculated by the representative value calculation unit 554 (step S681). The control unit 21 sets “1” for the node determined in step S681 and “0” for the other soft mask layer nodes. The control unit 21 calculates the back propagation of the fully-connected layer 552 (step S682).

The control unit 21 generates an image corresponding to the final layer of the feature quantity extraction unit 551. The control unit 21 performs predetermined weighting on the plurality of generated images, and calculates the weight given to the soft mask layer 553 by each part on the image. The control unit 21 defines the shape and position of the area of interest indicator 781 based on the heavy weight portion (step S683).

According to the present embodiment, it is possible to provide the diagnostic support system 10 that displays which part of the endoscope image 49 affects the diagnosis criteria prediction. By comparing the area of interest indicator 781 with the endoscope image 49 displayed in the endoscope image field 73, the user can understand which part of the endoscope image 49 contributed to the diagnosis criteria prediction. For example, when a part that is not photographed normally, such as a part with residue or a part with flare, contributes to the diagnosis criteria prediction, it can be determined that the user should ignore the displayed diagnosis criteria prediction.

By displaying the endoscope image field 73 and the area of interest field 78 separately, the user can observe the color and texture of the endoscope image 49 without being hindered by the area of interest indicator 781. By displaying the endoscope image field 73 and the area of interest field 78 on the same scale, the user can more intuitively grasp the positional relationship between the endoscope image 49 and the area of interest indicator 781.

### [First Modification]

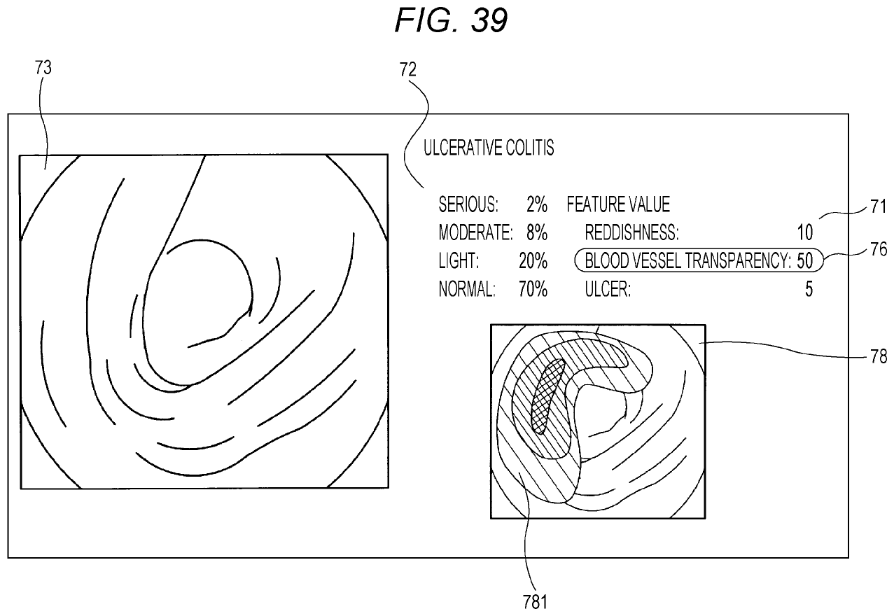

FIG. 39 is an explanatory diagram for explaining a screen display according to a first modification of the ninth embodiment. In the first modification, the endoscope image 49 and the area of interest indicator 781 are superimposed and displayed on the area of interest field 78. That is, the CPU 21 displays the same endoscope image 49 in the endoscope image field 73 and the area of interest field 78.

According to the present embodiment, the user can intuitively grasp the positional relationship between the endoscope image 49 and the area of interest indicator 781. In addition, by looking at the endoscope image field 73, the endoscope image 49 can be observed without being disturbed by the area of interest indicator 781.

### [Second Modification]

A second modification adds a function to display the area of interest indicator 781 to the diagnostic support system 10 of the sixth embodiment. Table 2 shows an example of the soft mask layer 553 of the first score learning model 611.

TABLE 2

| Output node number | Prediction contents |
|---|---|
| 1 | Normal |
| 2 | Presence of erythema |
| 3 | Presence of strong erythema |

For example, the probability that the reddishness state is “normal” is output from a first node of the soft mask layer 553. The probability of “presence of erythema” is output from a second node of soft mask layer 553. The probability of “presence of strong erythema” is output from a third node of soft mask layer 553.

The calculation in the representative value calculation unit 554 is not performed, and the output node of the soft mask layer 553 is output from the first model 61 as it is.

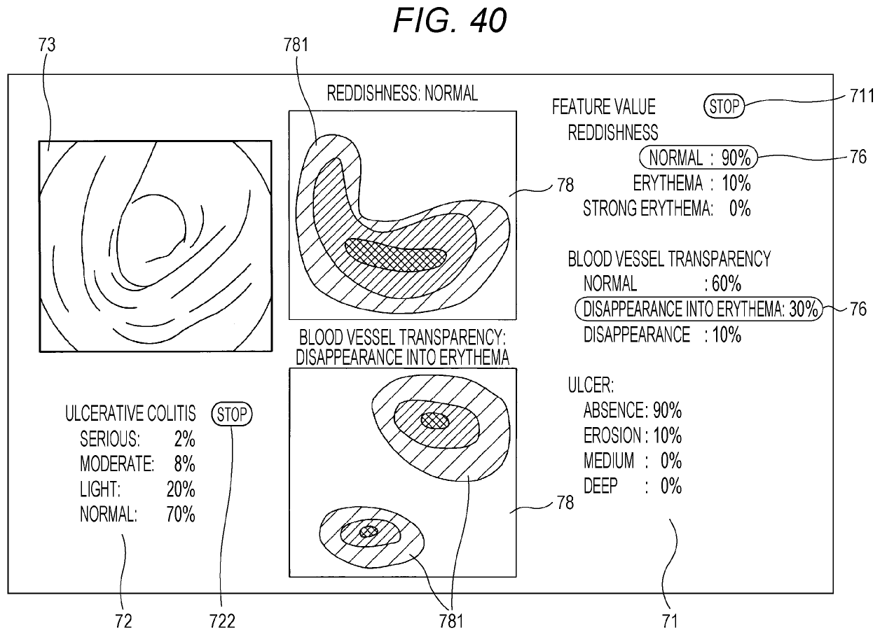

FIG. 40 is an explanatory diagram for explaining a screen display according to a second modification of the ninth embodiment. In the second modification, the probabilities of each category defined in the diagnostic criteria for disease in the first result field 71 are output.

In the example illustrated in FIG. 40, the select cursor 76 displays that the item of the “normal” in the first score of the first result field 71 and the item of the “disappearance into erythema” in the second score are selected by the user. Two areas of interest field 78 arranged vertically are displayed in the center of FIG. 40.

The first score will be described as an example. The control unit 21 sets “1” for the output node of the soft mask layer 553 corresponding to “normal” selected by the user, and “0” for the other output nodes. The control unit 21 performs the back propagation of the fully-connected layer 552 and generates the area of interest indicator 781 indicating the part that affects the determination that the probability of being “normal” is 90%.

The control unit 21 displays the area of interest indicator 781 regarding the probability that the “reddishness” is “normal” in the upper area of interest field 78. If the user changes the selection to the item of the “erythema”, the control unit 21 sets “1” for the output node of the soft mask layer 553 corresponding to the “erythema” and “0” for the other output nodes. The control unit 21 performs the back propagation of the fully-connected layer 552, and generates the area of interest indicator 781 indicating the part that affects the determination that the probability of being “erythema” is 10% and updates the screen.

The user may operate the select cursor 76 to select, for example, the item of the “normal” and the item of the “erythema” among the items of the “reddishness”. The user can check the part that affects the probability that the “reddishness” is “normal” in the area of interest field 78 and the part that affects the probability that the “reddishness” is “erythema”.

### [Third Modification]

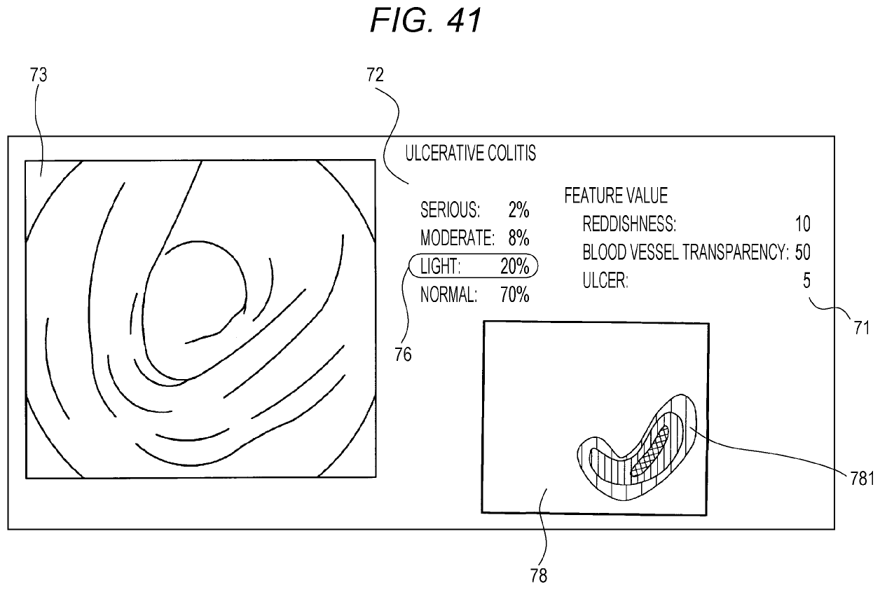

A third modification adds a function to display the area of interest indicator 781 for the item of diagnosis prediction. FIG. 41 is an explanatory diagram for explaining a screen display according to a third modification of the ninth embodiment.

In the example illustrated in FIG. 41, the select cursor 76 displays that the item of “light” in the second result field 72 is selected by the user. The area of interest indicator 781 which indicates the part that affects the determination that the ulcerative colitis is “light” is displayed in the area of interest field 78.

The user can confirm the part that affects the determination that the probability of being “light” is 20% by the area of interest indicator 781. The user can recheck the validity of the determination of “light” by the second model 62, for example, by further observing the location indicated by the area of interest indicator 781 from a different direction.

### Tenth Embodiment

The present embodiment relates to a diagnostic support system 10 that realizes an extraction unit 66 without using back propagation.

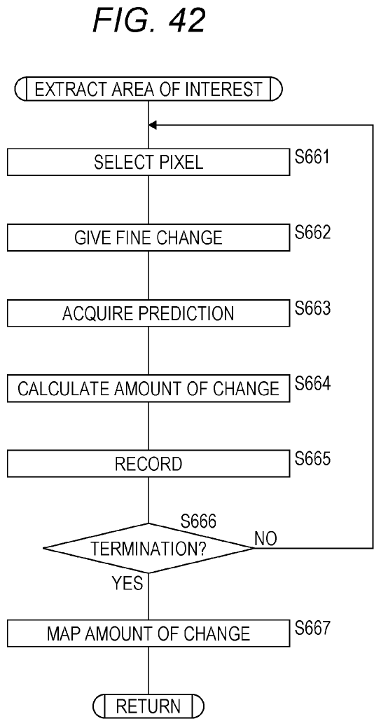

FIG. 42 is a flowchart for explaining a process flow of a subroutine of an area of interest extraction according to a tenth embodiment. The area of interest extraction subroutine is a subroutine that extracts an area of interest that affects a predetermined diagnosis criteria prediction from the endoscope image 49. The subroutine described with reference to FIG. 42 is executed in place of the subroutine described with reference to FIG. 38.

The control unit 21 selects one pixel from the endoscope image 49 (step S661). The control unit 21 imparts a minute change to a pixel selected in step S661 (step S662). The minute change is given by adding or subtracting 1 to any of the RGB (Red Green Blue) values of the selected pixel.

The control unit 21 inputs the changed endoscope image 49 to the first model 61 regarding the item selected by the user to obtain the diagnosis criteria prediction (step S663). The control unit 21 calculates the amount of change in the diagnosis criteria prediction, compared with the diagnosis criteria prediction acquired based on the endoscope image 49 before the change is given (step S664).

A pixel having a stronger influence on the diagnosis criteria prediction has a larger amount of change in the diagnosis criteria prediction due to a small change in the pixel. Therefore, the amount of change calculated in step S664 indicates the strength of the influence of the pixel on the diagnosis criteria prediction.

The control unit 21 records the amount of change calculated in step S664 in association with the position of the pixel selected in step S661 (step S665). The control unit 21 determines whether or not the process of all pixels is terminated (step S666). If it is determined that the process is not terminated (NO in step SS666), the control unit 21 returns to step S661.

If it is determined that the process is terminated (YES in step S666), the control unit 21 maps the amount of change based on the pixel position and the amount of change (step S667). The mapping is carried out, for example, by creating a heat map based on the magnitude of the amount of change, creating contour lines, or the like, and the shape and position of the area of interest indicator 781 indicating the area where the amount of change is large is determined. After that, the control unit 21 terminates the process.

Note that in step S661, the control unit 21 may select pixels every few pixels in both vertical and horizontal directions, for example. By thinning out the pixels, the process of the area of interest extraction subroutine can be speeded up.

The processes from step S651 to step S653 of FIG. 37 are executed instead of step S604 of the program at the time of the endoscope inspection of the third embodiment described with reference to FIG. 24, and the subroutine of the present embodiment may be executed in step S652. A function to display the area of interest indicator 781 can be added to the diagnostic support system 10 of the third embodiment.

The processes from step S651 to step S653 of FIG. 37 are executed instead of step S633 of the program at the time of the endoscope inspection of the forth embodiment described with reference to FIG. 28, and the subroutine of the present embodiment may be executed in step S652. A function to display the area of interest indicator 781 can be added to the diagnostic support system 10 of the fourth embodiment.

According to the present embodiment, even when the first model 61 does not have the soft mask layer 553 and the fully-connected layer 552, that is, even when a method other than the neural network model 53 is used, it is possible to provide the diagnostic support system 10 that can display the area of interest indicator 781.

The program of the present embodiment can also be applied to the area of interest extraction of the second model 62. In this case, in step S663 of the area of interest extraction subroutine described with reference to FIG. 42, the control unit 21 inputs the changed endoscope image 49 to the second model 62 to obtain the diagnosis prediction. In the following step S664, the control unit 21 compares the probability of being “light” acquired based on the endoscope image 49 before the change is given with the probability of being “light” acquired in step S664 to calculate the amount of change in the diagnosis prediction.

### Eleventh Embodiment

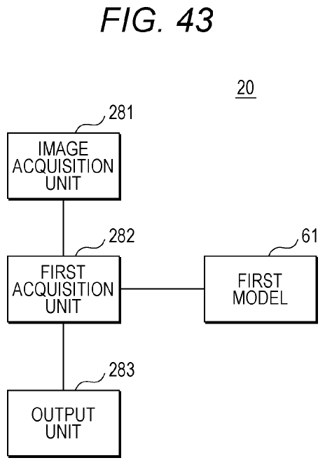

FIG. 43 is a functional block diagram of an information processing device 20 according to an eleventh embodiment. The information processing device 20 has an image acquisition unit 281, a first acquisition unit 282, and an output unit 283. The image acquisition unit 281 acquires the endoscope image 49.

The first acquisition unit 282 inputs the endoscope image 49 acquired by the image acquisition unit 281 to the first model 61 that outputs the diagnosis criteria prediction regarding diagnostic criteria of disease when the endoscope image 49 is input, and acquires the output diagnosis criteria prediction. The output unit 283 outputs the diagnosis criteria prediction acquired by the first acquisition unit 282 in association with the diagnosis criteria prediction regarding the state of the disease acquired based on the endoscope image 49.

### Twelfth Embodiment

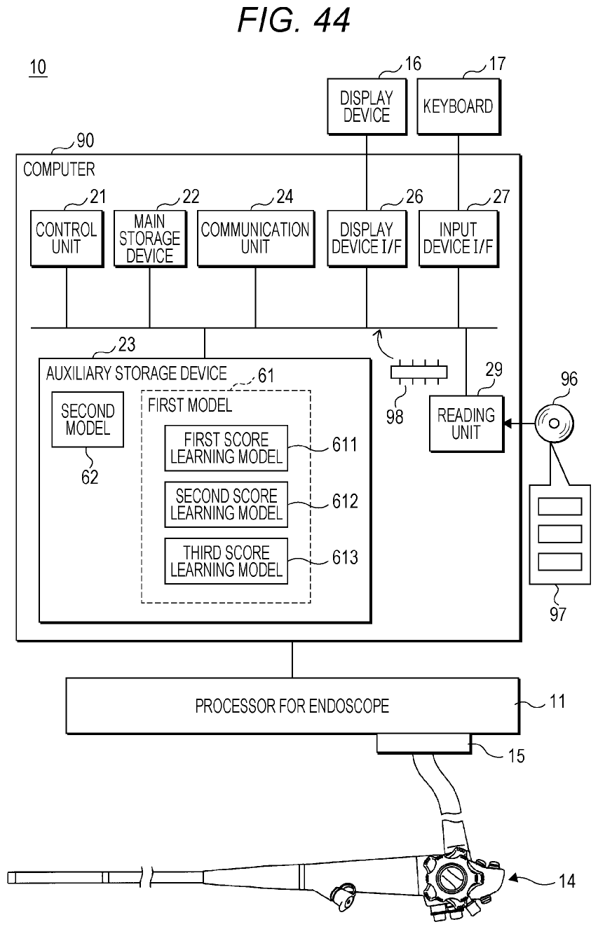

The present embodiment relates to a diagnostic support system 10 realized by operating a general-purpose computer 90 and a program 97 in combination. FIG. 44 is an explanatory diagram for explaining a configuration of the diagnostic support system 10 according to the twelfth embodiment. The description of the parts common to the first embodiment will be omitted.

The diagnostic support system 10 of the present embodiment includes a computer 90, a processor 11 for endoscope, and an endoscope 14. The computer 90 includes a control unit 21, a main storage device 22, an auxiliary storage device 23, a communication unit 24, a display device I/F 26, an input device I/F 27, a reading unit 29, and a bus. The computer 90 is an information device such as a general-purpose personal computer, a tablet, or a server computer.

The program 97 is recorded in a portable recording medium 96. The control unit 21 reads the program 97 via the reading unit 29 and stores the read program 97 in the auxiliary storage device 23. Further, the control unit 21 may read the program 97 stored in the semiconductor memory 98 such as a flash memory mounted in the computer 90. In addition, the control unit 21 may download the program 97 from the communication unit 24 and other server computers (not illustrated) connected via a network (not illustrated) and store the downloaded program 97 in the auxiliary storage device 23.

The program 97 is installed as a control program on the computer 90 and is loaded and executed on the main storage device 22. As a result, the computer 90, the processor 11 for endoscope, and the endoscope 14 function as the above-mentioned diagnostic support system 10.

### Thirteenth Embodiment

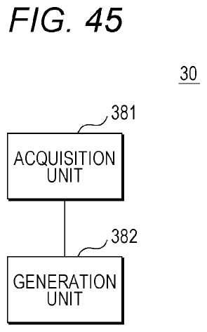

FIG. 45 is a functional block diagram of a server 30 according to a thirteenth embodiment. The server 30 has an acquisition unit 381 and a generation unit 382. The acquisition unit 381 acquires multiple sets of training data in which the endoscope image 49 and the determination result determined for the diagnostic criteria used in the diagnosis of disease are recorded in association with each other. The generation unit 382 uses the training data to generate the first model that outputs the diagnosis criteria prediction that predicts the diagnostic criteria of disease when the endoscope image 49 is input.

### Fourteenth Embodiment

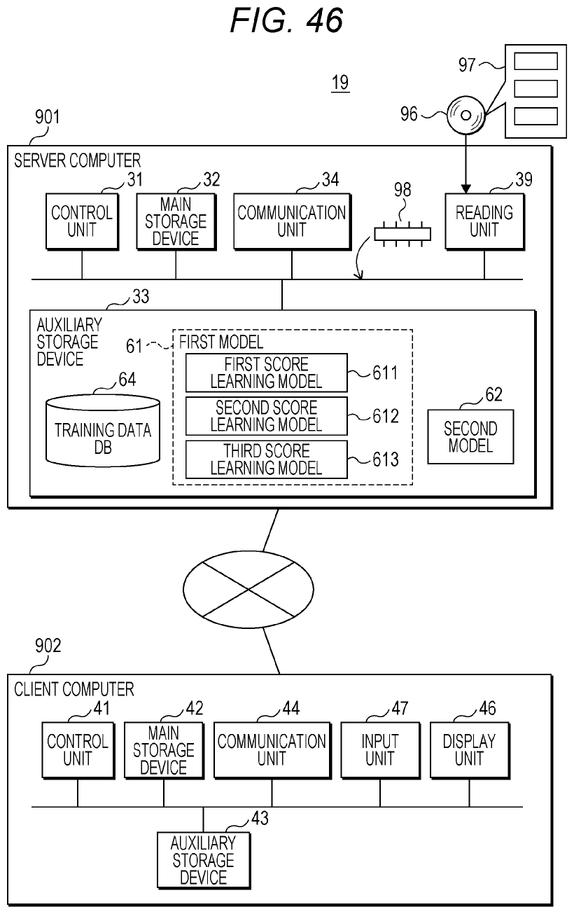

The present embodiment relates to a mode for realizing a model generation system 19 of the present embodiment by operating a general-purpose server computer 901, a client computer 902, and a program 97 in combination. FIG. 46 is an explanatory diagram for explaining a configuration of the model generation system 19 according to the fourteenth embodiment. The description of the parts common to the second embodiment will be omitted.

The model generation system 19 of the present embodiment includes a server computer 901 and a client computer 902. The server computer 901 includes a control unit 31, a main storage device 32, an auxiliary storage device 33, a communication unit 34, a reading unit 39, and a bus. The server computer 901 is a general-purpose personal computer, a tablet, a large computer, a virtual machine running on the large computer, a cloud computing system, or a quantum computer. The server computer 901 may be a plurality of personal computers or the like that perform distributed processing.

The client computer 902 includes a control unit 41, a main storage device 42, an auxiliary storage device 43, a communication unit 44, a display unit 46, an input unit 47, and a bus. The client computer 902 is an information device such as a general-purpose personal computer, a tablet, or a smartphone.

The program 97 is recorded in a portable recording medium 96. The control unit 31 reads the program 97 via the reading unit 39 and stores the read program 97 in the auxiliary storage device 33. Further, the control unit 31 may read the program 97 stored in the semiconductor memory 98 such as a flash memory mounted in the server computer 901. In addition, the control unit 31 may download the program 97 from the communication unit 24 and other server computers (not illustrated) connected via a network (not illustrated) and store the downloaded program 97 in the auxiliary storage device 33.

The program 97 is installed as a control program on the server computer 901 and is loaded and executed on the main storage device 22. The control unit 31 delivers the part of the program 97 executed by the control unit 41 to the client computer 902 via the network. The delivered program 97 is installed as a control program for the client computer 902, loaded into the main storage device 42, and executed.

As a result, the server computer 901 and the client computer 902 function as the above-mentioned diagnostic support system 10.

### Fifteenth Embodiment

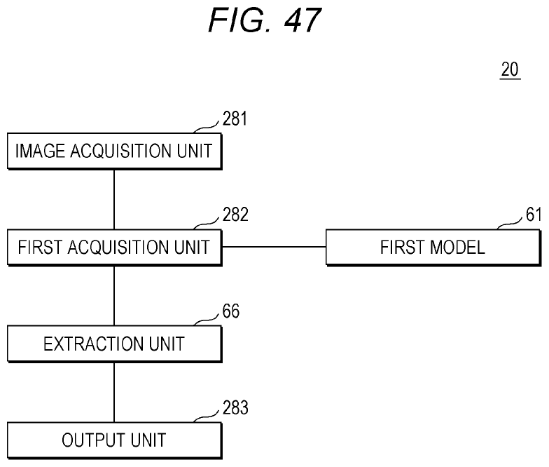

FIG. 47 is a functional block diagram of an information processing device 20 according to a fifteenth embodiment. The information processing device 20 has an image acquisition unit 281, a first acquisition unit 282, an extraction unit 66, and an output unit 283. The image acquisition unit 281 acquires the endoscope image 49.

The first acquisition unit 282 inputs the endoscope image 49 acquired by the image acquisition unit 281 to the first model 61 that outputs the diagnosis criteria prediction regarding diagnostic criteria of disease when the endoscope image 49 is input, and acquires the output diagnosis criteria prediction. The extraction unit 66 extracts an area that affects the diagnosis criteria prediction acquired by the first acquisition unit 282 from the endoscope image 49. The output unit 283 outputs the diagnosis criteria prediction acquired by the first acquisition unit 282, the indicator indicating the area extracted by the extraction unit 66, and the diagnosis prediction regarding the disease state acquired based on the endoscope image 49 in association with each other.

### Sixteenth Embodiment

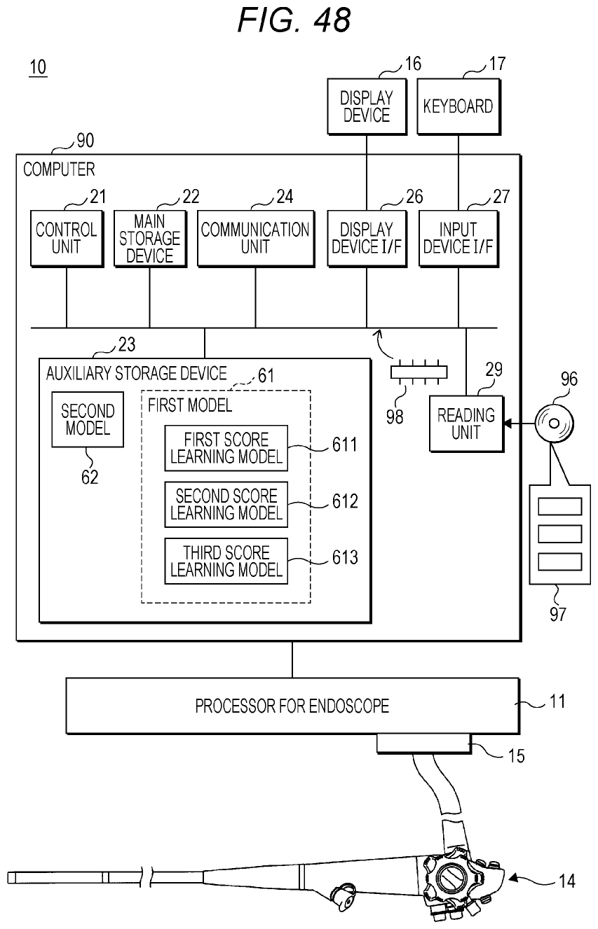

The present embodiment relates to a diagnostic support system 10 realized by operating a general-purpose computer 90 and a program 97 in combination. FIG. 48 is an explanatory diagram for explaining a configuration of the diagnostic support system 10 according to a sixteenth embodiment. The description of the parts common to the first embodiment will be omitted.

The diagnostic support system 10 of the present embodiment includes a computer 90, a processor 11 for endoscope, and an endoscope 14. The computer 90 includes a control unit 21, a main storage device 22, an auxiliary storage device 23, a communication unit 24, a display device I/F 26, an input device I/F 27, a reading unit 29, and a bus. The computer 90 is an information device such as a general-purpose personal computer, a tablet, or a server computer.

The program 97 is recorded in a portable recording medium 96. The control unit 21 reads the program 97 via the reading unit 29 and stores the read program 97 in the auxiliary storage device 23. Further, the control unit 21 may read the program 97 stored in the semiconductor memory 98 such as a flash memory mounted in the computer 90. In addition, the control unit 21 may download the program 97 from the communication unit 24 and other server computers (not illustrated) connected via a network (not illustrated) and store the downloaded program 97 in the auxiliary storage device 23.

The program 97 is installed as a control program on the computer 90 and is loaded and executed on the main storage device 22. As a result, the computer 90, the processor 11 for endoscope, and the endoscope 14 function as the above-mentioned diagnostic support system 10.

The technical features (constituent requirements) described in each embodiment can be combined with each other, and a new technical feature can be formed by the combination.

The embodiments disclosed this time should be considered to be exemplary in all respects without being limited. The scope of the present invention is indicated by the scope of claims, not the above-mentioned meaning, and is intended to include all modifications within the meaning and scope equivalent to the claims.

### APPENDIX 1

An information processing device, including:

an image acquisition unit that acquires an endoscope image;

a first acquisition unit that inputs the endoscope image acquired by the image acquisition unit to a first model that outputs diagnosis criteria prediction regarding diagnostic criteria of disease when the endoscope image is input, and acquires the output diagnosis criteria prediction; and

an output unit that outputs the diagnosis criteria prediction acquired by the first acquisition unit in association with the diagnosis prediction regarding a state of the disease acquired based on the endoscope image.

### APPENDIX 2

The information processing device described in appendix 1, in which the first acquisition unit acquires diagnosis criteria predictions of each item from a plurality of first models that output each diagnosis criteria prediction of the plurality of items included in the diagnostic criteria of the disease.

### APPENDIX 3

The information processing device described in appendix 1 or 2, in which the first model is a learning model generated by machine learning.

### APPENDIX 4

The information processing device described in appendix 1 or 2, in which the first model outputs a numerical value calculated based on the endoscope image acquired by the image acquisition unit.

### APPENDIX 5

The information processing device described in any one of appendixes 1 to 4, further including: a first reception unit that receives an operation stop instruction of the first acquisition unit.

### APPENDIX 6

The information processing device described in any one of appendixes 1 to 5, in which

the diagnosis prediction is a diagnosis prediction output by inputting the endoscope image acquired by the image acquisition unit to a second model that outputs the diagnosis prediction of the disease when the endoscope image is input.

### APPENDIX 7

The information processing device described in appendix 6, in which the second model is a learning model generated by machine learning.

### APPENDIX 8

The information processing device described in appendix 6 or 7, in which

the second model includes

a neural network model that includes an input layer to which the endoscope image is input,

an output layer that outputs the diagnosis prediction of the disease, and

an intermediate layer in which parameters are learned by multiple sets of training data recorded by associating the endoscope image with the diagnosis prediction, and

the first model outputs a diagnosis criteria prediction based on a feature quantity acquired from a predetermined node of the intermediate layer.

### APPENDIX 9

The information processing device described in appendix 6 or 7, in which

the second model outputs an area prediction regarding a legion region including the disease when the endoscope image is input,

the first model outputs the diagnosis criteria prediction regarding the diagnostic criteria of the disease when the endoscope image of the legion region is input, and the first acquisition unit inputs a part corresponding to the area prediction output from the second model in the endoscope image acquired by the image acquisition unit to the first model, and acquires the output diagnosis criteria prediction.

### APPENDIX 10

The information processing device described in any one of appendixes 6 to 9, further including: a second reception unit that receives an instruction to stop the acquisition of the diagnosis prediction.

### APPENDIX 11

The information processing device described in any one of appendixes 6 to 10, in which the output unit outputs the endoscope image acquired by the image acquisition unit.

### APPENDIX 12

The information processing device described in any one of appendixes 1 to 11, in which the image acquisition unit acquires the endoscope image photographed during endoscope inspection in real time, and

the output unit performs an output in synchronization with the acquisition of the endoscope image by the image acquisition unit.

### APPENDIX 13

A processor for endoscope, including:

an endoscope connection unit to which an endoscope is connected;

an image generation unit that generates an endoscope image based on a video signal acquired from the endoscope connected to the endoscope connection unit;

a first acquisition unit that inputs the endoscope image generated by the image generation unit to a first model that outputs diagnosis criteria prediction regarding diagnostic criteria of disease when the endoscope image is input, and acquires the output diagnosis criteria prediction; and

an output unit that outputs the diagnosis criteria prediction acquired by the first acquisition unit in association with the diagnosis prediction regarding a state of the disease acquired based on the endoscope image.

### APPENDIX 14

An information processing method that causes a computer to execute the following processes of:

acquiring an endoscope image;

inputting the acquired endoscope image to a first model that outputs diagnosis criteria prediction regarding diagnostic criteria of disease when the endoscope image is input, and acquiring the output diagnosis criteria prediction; and

outputting the acquired diagnosis criteria prediction in association with the diagnosis prediction regarding a state of the disease acquired based on the endoscope image.

### APPENDIX 15

A program that causes a computer to execute the following processes of:

acquiring an endoscope image;

inputting the acquired endoscope image to a first model that outputs diagnosis criteria prediction regarding diagnostic criteria of disease when the endoscope image is input, and acquiring the output diagnosis criteria prediction; and

outputting the acquired diagnosis criteria prediction in association with the diagnosis prediction regarding a state of the disease acquired based on the endoscope image.

### APPENDIX 16

A model generation method, including:

acquiring multiple sets of training data in which an endoscope image and a determination result determined for diagnostic criteria used in a diagnosis of disease are recorded in association with each other

and

using the training data to generate a first model that outputs a diagnosis criteria prediction that predicts the diagnostic criteria of disease when the endoscope image is input.

### APPENDIX 17

The model generation method described in appendix 16, in which

the training data includes a determination result determined for each of a plurality of diagnostic criteria items included in the diagnostic criteria, and

the first model is generated corresponding to each of the plurality of diagnostic criteria items.

### APPENDIX 18

The model generation method described in appendix 16 or 17, in which the first model is generated by deep learning that adjusts parameters of an intermediate layer so that the acquired determination result is output from an output layer when the acquired endoscope image is input to the input layer.

### APPENDIX 19

The model generation method described in appendix 16 or 17, in which

the first model is generated by

inputting the endoscope image in the acquired training data to a neural network model that outputs the diagnosis prediction of the disease when the endoscope image is input,

acquiring a plurality of feature quantities related to the input endoscope image from a node that constitutes the intermediate layer of the neural network model,

selecting a feature quantity having a high correlation with the determination result associated with the endoscope image from the plurality of acquired feature quantities, and

defining a calculation method for calculating the score based on the selected feature quantity by regression analysis of the selected feature quantity and the score obtained by quantifying the determination result.

### APPENDIX 20

The model generation method described in appendix 16 or 17, in which

the first model is generated by

extracting a plurality of feature quantities from the acquired endoscope image,

selecting a feature quantity having a high correlation with the determination result associated with the endoscope image from the plurality of extracted feature quantities, and

defining a calculation method for calculating the score based on the selected feature quantity by regression analysis of the selected feature quantity and the score obtained by quantifying the determination result.

### APPENDIX 21

The model generation method described in any one of appendixes 16 to 20, in which

the disease is ulcerative colitis, and

the diagnosis criteria prediction is a prediction of the endoscope image regarding reddishness, the blood vessel transparency, or seriousness of ulcer.

### APPENDIX 22

A program that causes a computer to execute a process of generating a first model:

by acquiring multiple sets of training data in which an endoscope image and a determination result determined for diagnostic criteria used in a diagnosis of disease are recorded in association with each other,

inputting the endoscope image in the acquired training data to a neural network model that outputs the diagnosis prediction of the disease when the endoscope image is input,

acquiring a plurality of feature quantities related to the input endoscope image from a node that constitutes the intermediate layer of the neural network model,

recording the plurality of acquired feature quantities by associating a determination result associated with the input endoscope image with a quantified score,

selects a feature quantity having a high correlation with the score based on the correlation between each of the plurality of recorded feature quantities and the score, and

outputs the diagnosis criteria prediction predicted for the diagnostic criteria of the disease when an endoscope image is input by defining a calculation method for calculating the score based on the selected feature quantity by regression analysis of the selected feature quantity and the score.

### APPENDIX 23

An information processing device, including:

an image acquisition unit that acquires an endoscope image;

a first acquisition unit that inputs the endoscope image acquired by the image acquisition unit to a first model that outputs diagnosis criteria prediction regarding diagnostic criteria of disease when the endoscope image is input, and acquires the output diagnosis criteria prediction;

an extraction unit that extracts an area that affects the diagnosis criteria prediction acquired by the first acquisition unit from the endoscope image; and

an output unit that outputs the diagnosis criteria prediction acquired by the first acquisition unit, an indicator indicating the area extracted by the extraction unit, and the diagnosis prediction regarding a state of the disease acquired based on the endoscope image in association with each other.

### APPENDIX 24

The information processing device described in appendix 23, in which

the first acquisition unit acquires the diagnosis criteria predictions of each item from a plurality of first models that output each diagnosis criteria prediction of a plurality of items related to the diagnostic criteria of the disease, and

the information processing device further includes a reception unit that receives a selection item from the plurality of items, and

the extraction unit extracts an area that affects the diagnosis criteria prediction regarding the selection item accepted by the reception unit.

### APPENDIX 25

The information processing device described in appendix 23 or 24, in which the output unit outputs the endoscope image and the indicator side by side.

### APPENDIX 26

The information processing device described in appendix 23 or 24, in which the output unit outputs the endoscope image and the indicator in an overlapping manner.

### APPENDIX 27

The information processing device described in any one of appendixes 23 to 26, further including: a stop reception unit that receives an operation stop instruction of the extraction unit.

### APPENDIX 28

The information processing device described in any one of appendixes 23 to 27, further including:

a second acquisition unit that inputs the endoscope image acquired by the image acquisition unit to a second model that outputs the diagnosis prediction of the disease when the endoscope image is input and acquires the output diagnosis prediction,

wherein the output unit outputs the diagnosis criteria prediction acquired by the second acquisition unit, the diagnosis prediction acquired by the first acquisition unit, and the indicator.

### APPENDIX 29

An information processing device, including:

an image acquisition unit that acquires an endoscope image;

a second acquisition unit that inputs the endoscope image acquired by the image acquisition unit to a second model that outputs a diagnosis prediction of disease when the endoscope image is input and acquires the output diagnosis prediction;

an extraction unit that extracts an area that affects the diagnosis criteria prediction acquired by the second acquisition unit from the endoscope image; and

an output unit that outputs the diagnosis prediction acquired by the second acquisition unit in association with the indicator indicating the region extracted by the extraction unit.

### APPENDIX 30

A processor for endoscope, including:

an endoscope connection unit to which an endoscope is connected;

an image generation unit that generates an endoscope image based on a video signal acquired from the endoscope connected to the endoscope connection unit;

a first acquisition unit that inputs a video signal acquired from the endoscope to a first model that outputs diagnosis criteria prediction regarding diagnostic criteria of disease when the video signal acquired from the endoscope image is input, and acquires the output diagnosis criteria prediction,

an extraction unit that extracts an area that affects the diagnosis criteria prediction acquired by the first acquisition unit from the endoscope image; and

an output unit that outputs the diagnosis criteria prediction acquired by the first acquisition unit, an indicator indicating the area extracted by the extraction unit, and the diagnosis prediction regarding a state of the disease acquired based on the endoscope image in association with each other.

### APPENDIX 31

An information processing method that causes a computer to execute the following processes of:

acquiring an endoscope image;

inputting the acquired endoscope image to a first model that outputs diagnosis criteria prediction regarding diagnostic criteria of disease when the endoscope image is input, and acquiring the output diagnosis criteria prediction;

extracting an area that affects the acquired diagnosis criteria prediction from the endoscope image; and

outputting the acquired diagnosis criteria prediction, the indicator indicating the extracted area, and the diagnosis prediction regarding a state of the disease acquired based on the endoscope image in association with each other.

### APPENDIX 32

A program that causes a computer to execute the following processes of:

acquiring an endoscope image;

inputting the acquired endoscope image to a first model that outputs diagnosis criteria prediction regarding diagnostic criteria of disease when the endoscope image is input, and acquiring the output diagnosis criteria prediction;

extracting an area that affects the acquired diagnosis criteria prediction from the endoscope image; and

outputting the acquired diagnosis criteria prediction, the indicator indicating the extracted area, and the diagnosis prediction regarding a state of the disease acquired based on the endoscope image in association with each other.

### REFERENCE SIGNS LIST

10 Diagnostic support system
11 Processor for endoscope
12 Endoscope connection unit
14 Endoscope
141 Image sensor
142 Insertion unit
15 Endoscope connector
16 Display device
161 First display device
162 Second display device
17 Keyboard
19 Model generation system
20 Information processing device
21 Control unit
22 Main storage device
23 Auxiliary storage device
24 Communication unit
26 Display device I/F
27 Input device I/F
281 Image acquisition unit
282 First acquisition unit
283 Output unit
29 Reading unit
30 Server
31 Control unit
32 Main storage device
33 Auxiliary storage device
34 Communication unit
381 Acquisition unit
382 Generation unit
39 Reading unit
40 Client
41 Control unit
42 Main storage device
43 Auxiliary storage device
44 Communication unit
46 Display unit
47 Input unit
49 Endoscope image
53 Neural network model
531 Input layer
532 Intermediate layer
533 Output layer
551 Feature quantity extraction unit
552 Fully-connected layer
553 Soft mask layer
554 Representative value calculation unit
61 First model
611 First score learning model
612 Second score learning model
613 Third score learning model
62 Second model
63 Converter
631 First converter
632 Second converter
633 Third converter
64 Training data DB
65 Feature quantity
651 First feature quantity
652 Second feature quantity
653 Third feature quantity
66 Extraction unit
71 First result field
711 First stop button
72 Second result field
722 Second stop button
73 Endoscope image field
74 Legion region
75 Warning field
76 Select cursor
78 Area of interest field
781 Area of interest indicator (indicator)
81 First input field
811 First score input field
812 Second score input field
813 Third score input field
82 Second input field
86 Patient ID field
87 Disease name field
88 Model button
89 Next button
90 Computer
901 Server computer
902 Client computer
96 Portable recording medium
97 Program
98 Semiconductor memory

1. An information processing device, comprising:
an image acquisition unit that acquires an endoscope image;
a first acquisition unit that inputs the endoscope image acquired by the image acquisition unit to a first model that outputs diagnosis criteria prediction regarding diagnostic criteria of disease when the endoscope image is input, and acquires the output diagnosis criteria prediction; and
an output unit that outputs the diagnosis criteria prediction acquired by the first acquisition unit in association with the diagnosis prediction regarding a state of the disease acquired based on the endoscope image.

2. The information processing device according to claim 1, wherein
the first acquisition unit acquires the diagnosis criteria predictions of each item from a plurality of first models that output each diagnosis criteria prediction of a plurality of items included in the diagnostic criteria of the disease.

3. The information processing device according to claim 1, wherein the first model is a learning model generated by machine learning.

4. The information processing device according to claim 1, wherein the first model outputs a numerical value calculated based on the endoscope image acquired by the image acquisition unit.

5. The information processing device according to claim 1, further comprising: a first reception unit that receives an operation stop instruction of the first acquisition unit.

6. The information processing device according to claim 1, wherein the diagnosis prediction is a diagnosis prediction output by inputting the endoscope image acquired by the image acquisition unit to a second model that outputs the diagnosis prediction of the disease when the endoscope image is input.

7. The information processing device according to claim 6, wherein the second model is a learning model generated by machine learning.

8. The information processing device according to claim 6, wherein
the second model includes
a neural network model that includes an input layer to which the endoscope image is input,
an output layer that outputs the diagnosis prediction of the disease, and
an intermediate layer in which parameters are learned by multiple sets of training data recorded by associating the endoscope image with the diagnosis prediction, and
the first model outputs a diagnosis criteria prediction based on a feature quantity acquired from a predetermined node of the intermediate layer.

9. The information processing device according to claim 6, wherein
the second model outputs an area prediction regarding a legion region including the disease when the endoscope image is input,
the first model outputs the diagnosis criteria prediction regarding the diagnostic criteria of the disease when the endoscope image of the legion region is input, and
the first acquisition unit inputs a part corresponding to the area prediction output from the second model in the endoscope image acquired by the image acquisition unit to the first model, and acquires the output diagnosis criteria prediction.

10. The information processing device according to claim 6, further comprising: a second reception unit that receives an instruction to stop the acquisition of the diagnosis prediction.

11. The information processing device according to claim 1, wherein the image acquisition unit acquires the endoscope image photographed during endoscope inspection in real time, and
the output unit performs an output in synchronization with the acquisition of the endoscope image by the image acquisition unit.

12. An information processing device, comprising:
an image acquisition unit that acquires an endoscope image;
a first acquisition unit that inputs the endoscope image acquired by the image acquisition unit to a first model that outputs diagnosis criteria prediction regarding diagnostic criteria of disease when the endoscope image is input, and acquires the output diagnosis criteria prediction;
an extraction unit that extracts an area that affects the diagnosis criteria prediction acquired by the first acquisition unit from the endoscope image; and
an output unit that outputs the diagnosis criteria prediction acquired by the first acquisition unit, an indicator indicating the area extracted by the extraction unit, and the diagnosis prediction regarding a state of the disease acquired based on the endoscope image in association with each other.

13. The information processing device according to claim 12, wherein
the first acquisition unit acquires the diagnosis criteria predictions of each item from a plurality of first models that output each diagnosis criteria prediction of a plurality of items related to the diagnostic criteria of the disease, and
the information processing device further includes a reception unit that receives a selection item from the plurality of items, and
the extraction unit extracts an area that affects the diagnosis criteria prediction regarding the selection item accepted by the reception unit.

14. A model generation method, comprising:
acquiring multiple sets of training data in which an endoscope image and a determination result determined for diagnostic criteria used in a diagnosis of disease are recorded in association with each other; and
using the training data to generate a first model that outputs a diagnosis criteria prediction that predicts the diagnostic criteria of disease when the endoscope image is input.

15. The model generation method according to claim 14, wherein the training data includes a determination result determined for each of a plurality of diagnostic criteria items included in the diagnostic criteria, and
the first model is generated corresponding to each of the plurality of diagnostic criteria items.
